(19) United States
(12) Patent Application Publication
(10) Pub. No.: US 2008/0108586 A1
(43) Pub. Date: May 8, 2008

(54) COMBINATION THERAPY FOR HUMAN IMMUNODEFICIENCY VIRUS INFECTION

(75) Inventors: Paul A. Friedman, Villanova, PA (US); Susan Erickson-Viitanen, West Chester, PA (US)

Correspondence Address: FISH & RICHARDSON PC, P.O. BOX 1022, MINNEAPOLIS, MN, 55440-1022, US

(73) Assignee: Incyte Corporation, Wilmington, DE (US)

(21) Appl. No.: 11/850,238

(22) Filed: Sep. 5, 2007

Related U.S. Application Data
(60) Provisional application No. 60/842,544, filed on Sep. 6, 2006.

(51) Int. Cl. A61K 31/7028; A61K 31/675; A61K 31/536; A61P 31/18

(52) U.S. Cl. 514/49; 514/81; 514/230.5

(57) ABSTRACT

The present invention is directed to combination therapies for treatment of Human Immunodeficiency Virus (HIV) infection comprising administration of a CCR5 antagonist in combination with other therapeutic agents.

### CROSS REFERENCE TO RELATED APPLICATIONS

This application claims the benefit of U.S. Ser. No. 60/842,544, filed Sep. 6, 2006, the disclosure of which is incorporated herein by reference in its entirety.

### TECHNICAL FIELD

This invention relates to combination therapies for treatment of Human Immunodeficiency Virus (HIV) infection comprising administration of a CCR5 antagonist in combination with other therapeutic agents.

### BACKGROUND

HIV infection is a major worldwide medical problem. The number of new cases of HIV and AIDS (acquired immunodeficiency syndrome) continues to rise rapidly. Currently available drugs for the treatment of HIV include nucleoside reverse transcriptase inhibitors (NRTI's) or approved single pill combinations, such as zidovudine (AZT), didanosine (ddI), stavudine (d4T), lamivudine (3TC), zalcitabine (ddC), abacavir succinate, tenofovir (including tenofovir disoproxil and tenofovir disoproxil fumarate salt, emtricitabine, COMBIVIR™. (contains 3TC and AZT), TRIZIVIR™ (contains abacavir, 3TC and AZT), TRUVADA™ (contains tenofovir and emtricitabine), EPZICOM™ (contains abacavir and 3TC); non-nucleoside reverse transcriptase inhibitors (NNRTIs), such as TMC-125, nevirapine, delavirdine and efavirenz; peptidomimetic protease inhibitors or approved formulations such as saquinavir, indinavir, ritonavir, nelfinavir, amprenavir, atazanavir, fosamprenavir, KALETRA™ (contains lopinavir and ritonavir); and at least one fusion inhibitor, which is enfuvirtide.

Zidovudine, known commonly as AZT is an antiretroviral drug, classified as a NRTI. It was the first drug approved for treatment of HIV. It is a thymidine analog and has the chemical name 1-[(2R,4S,5S)-4-azido-5-(hydroxymethyl)oxolan-2-yl]-5-methyl-pyrimidine-2,4-dione [30516-87-1].

Lamivudine, known commonly as 3TC is another NTRI antiretroviral drug used to treat HIV. It has the chemical name L-2′,3′-dideoxy-3′-thiacytidine [14678-17-4]. Lamivudine is an analog of cytidine. It can inhibit both types (1 and 2) of HIV reverse transcriptase and also the reverse transcriptase of hepatitis B. It needs to be phosphorylated to its triphosphate form before it is active. 3TC-triphosphate also inhibits cellular DNA polymerase. Lamivudine is administered orally, and it is rapidly absorbed with a bio-availability of over 80%. Some research suggests that lamivudine can cross the blood-brain barrier. Lamivudine is often given in combination with zidovudine, with which it is highly synergistic. Lamivudine treatment has been shown to restore zidovudine sensitivity of previously resistant HIV.

Emtricitabine is another cytidine analog NTRI drug used in HIV treatment. It has the chemical name (4-amino-5-fluoro-1-[2-(hydroxylmethyl)-1,3-oxathiolan-5-yl]-pyrimidin-2-one [143491-57-0]. By interfering with reverse transcriptase, which is central to the replication of HIV, emtricitabine can help to lower the amount of HIV, or “viral load”, in a patient's body and can indirectly increase the number of immune system cells (called T cells or CD4+ T-cells). Both of these changes are associated with healthier immune systems and decreased likelihood of serious illness. Emtricitabine is also marketed in a fixed-dose combination with tenofovir (VIREAD®) under the brand name TRUVADA®. A fixed-dose triple combination of emtricitabine, tenofovir and efavirenz (SUSTIVA®, marketed by Bristol-Myers Squibb) has been approved by the FDA on Jul. 12, 2006 under the brand name ATRIPLA®.

Tenofovir belongs to a class of antiretroviral drugs known as nucleotide analogue reverse transcriptase inhibitors (NtRTIs), which block reverse transcriptase. It has the chemical name 1-(6-aminopurin-9-yl)propan-2-yloxymethylphosphonic acid [147127-20-6]. Tenofovir disoproxil fumarate (a prodrug of tenofovir) is a fumaric acid salt of bis-isopropoxycarbonyloxymethyl ester derivative of tenofovir. It is sold under the brand name VIREAD® as tablets are for oral administration, each tablet containing 300 mg of tenofovir disoproxil fumarate, which is equivalent to 245 mg of tenofovir disoproxil. In vivo, tenofovir disoproxil fumarate is converted to tenofovir, an acyclic nucleoside phosphonate (nucleotide) analog of adenosine 5′-monophosphate. Tenofovir exhibits activity against HIV reverse transcriptase. As discussed above, tenofovir is also marketed in at least two fixed dose combinations with other antiretroviral drugs.

Efavirenz falls in the non-nucleoside reverse transcriptase inhibitor (NNRTI) class of antiretroviral drugs. Both nucleoside and non-nucleoside RTIs inhibit the same target, the reverse transcriptase enzyme, an essential viral enzyme which transcribes viral RNA into DNA. Unlike NRTIs, which bind at the enzyme's active site, NNRTIs bind within a pocket termed the NNRTI pocket. Efavirenz has the chemical name 8-chloro-5-(2-cyclopropyl-ethynyl)-5-(trifluoromethyl)-4-oxa-2-azabicyclo [4.4.0]deca-7,9,11-trien-3-one [154598-52-4]. See also, Chearskul et al., Indian J Pediatr., 2006, 73(4), 335-41) and Bartlett et al., Ann Intern Med. 1996, 125(3), 161-72).

Chemotactic cytokines (leukocyte chemoattractant/activating factors) also known as chemokines, also known as intercrines and SIS cytokines are a group of inflammatory/immunomodulatory polypeptide factors of molecular weight 6-15 kDa that are released by a wide variety of cells such as macrophages, monocytes, eosinophils, neutrophils, fibroblasts, vascular endothelial cells, smooth muscle cells, and mast cells, at inflammatory sites (reviewed in Luster, New Eng. J Med., 338, 436-445 (1998) and Rollins, Blood, 90, 909-928 (1997)). Also, chemokines have been described in Oppenheim, J. J. et al., Annu. Rev. Immunol., 9:617-648 (1991); Schall and Bacon, Curr. Opin. Immunol., 6:865-873 (1994); Baggiolini, M., et al., and Adv. Immunol., 55:97-179 (1994). Chemokines have the ability to stimulate directed cell migration, a process known as chemotaxis. Each chemokine contains four cysteine residues (C) and two internal disulfide bonds. Chemokines can be grouped into two subfamilies, based on whether the two amino terminal cysteine residues are immediately adjacent (CC family) or separated by one amino acid (CXC family). These differences correlate with the organization of the two subfamilies into separate gene clusters. Within each gene cluster, the chemokines typically show sequence similarities between 25 to 60%. The CXC chemokines, such as interleukin-8 (IL-8), neutrophil-activating protein-2 (NAP-2) and melanoma growth stimulatory activity protein (MGSA) are chemotactic primarily for neutrophils and T lymphocytes, whereas the CC chemokines, such as RANTES, MIP-1α, MIP-1β, the monocyte chemotactic proteins (MCP-1, MCP-2, MCP-3, MCP-4, and MCP-5) and the eotaxins (−1 and −2) are chemotactic for, among other cell types, macrophages, T lymphocytes, eosinophils, dendritic cells, and basophils. There also exist the chemokines lymphotactin-1, lymphotactin-2 (both C chemokines), and fractalkine (a CXXXC chemokine) that do not fall into either of the major chemokine subfamilies.

The chemokines bind to specific cell-surface receptors belonging to the family of G-protein-coupled seven-transmembrane-domain proteins (reviewed in Horuk, Trends Pharm. Sci., 15, 159-165 (1994)) which are termed “chemokine receptors.” On binding their cognate ligands, chemokine receptors transduce an intracellular signal through the associated trimeric G proteins, resulting in, among other responses, a rapid increase in intracellular calcium concentration, changes in cell shape, increased expression of cellular adhesion molecules, degranulation, and promotion of cell migration.

Genes encoding receptors of specific chemokines have been cloned, and it is known that these receptors are G protein-coupled seven-transmembrane receptors present on various leukocyte populations. So far, at least five CXC chemokine receptors (CXCR1-CXCR5) and eight CC chemokine receptors (CCR1-CCR8) have been identified. For example IL-8 is a ligand for CXCR1 and CXCR2, MIP-1α is that for CCR1 and CCR5, and MCP-1 is that for CCR2A and CCR2B (for reference, see for example, Holmes, W. E., et al., Science 1991, 253, 1278-1280; Murphy P. M., et al., Science, 253, 1280-1283; Neote, K. et al, Cell, 1993, 72, 415-425; Charo, I. F., et al., Proc. Natl. Acad. Sci. USA, 1994, 91, 2752-2756; Yamagami, S., et al., Biochem. Biophys. Res. Commun., 1994, 202, 1156-1162; Combadier, C., et al., The Journal of Biological Chemistry, 1995, 270, 16491-16494, Power, C. A., et al., J. Biol. Chem., 1995, 270, 19495-19500; Samson, M., et al., Biochemistry, 1996, 35, 3362-3367; Murphy, P. M., Annual Review of Immunology, 1994, 12, 592-633). It has been reported that lung inflammation and granuroma formation are suppressed in CCR1-deficient mice (see Gao, J.-L., et al., J. Exp. Med., 1997, 185, 1959; Gerard, C., et al., J. Clin. Invest., 1997, 100, 2022), and that recruitment of macrophages and formation of atherosclerotic lesion decreased in CCR2-deficient mice (see Boring, L., et al., Nature, 1998, 394, 894; Kuziel, W. A., et al., Proc. Natl. Acad. Sci., USA, 1997, 94, 12053; Kurihara, T., et al., J. Exp. Med., 1997, 186, 1757; Boring, L., et al., J. Clin. Invest., 1997, 100, 2552).

Chemokine receptors are also known as coreceptors for viral entry leading to viral infection such as, for example, HIV infection. Reverse transcription and protein processing are the classic steps of the viral life cycle which antiretroviral therapeutic agents are designed to block. Although many new drugs that are believed to block viral entry hold promise, there is currently no agent to which HIV-1 has not been able to acquire resistance. Multiple rounds of viral replication are required to generate the genetic diversity that forms the basis of resistance. Combination therapy in which replication is maximally suppressed remains a cornerstone of treatment with entry inhibitors, as with other agents. The targeting of multiple steps within the viral entry process is believed to have the potential for synergy (Starr-Spires et al., Clin. Lab. Med., 2002, 22(3), 681.)

HIV-1 entry into CD4(+) cells requires the sequential interactions of the viral envelope glycoproteins with CD4 and a coreceptor such as the chemokine receptors CCR5 and CXCR4. A plausible approach to blocking this process is to use small molecule antagonists of coreceptor function. The TAK-779 molecule is one such antagonist of CCR5 that acts to prevent HIV-1 infection. TAK-779 inhibits HIV-1 replication at the membrane fusion stage by blocking the interaction of the viral surface glycoprotein gp120 with CCR5. The binding site for TAK-779 on CCR5 is located near the extracellular surface of the receptor, within a cavity formed between transmembrane helices 1, 2, 3, and 7 (Dragic et al., Proc. Natl. Acad. Sci. USA, 2000, 97(10), 5639).

The chemokine receptors CXCR4 and CCR5 are believed to be used as co-receptors by the T cell-tropic (X4) and macrophage-tropic (R5) HIV-1 strains, respectively, for entering their host cells. Propagation of R5 strains of HIV-1 on CD4 lymphocytes and macrophages requires expression of the CCR5 coreceptor on the cell surface. Individuals lacking CCR5 (CCR5Delta 32 homozygous genotype) are phenotypically normal and resistant to infection with HIV-1. Viral entry can be inhibited by the natural ligands for CXCR4 (the CXC chemokine SDF-1) and CCR5 (the CC chemokines RANTES, MIP-1alpha and MIP-1beta). The first non-peptidic compound that interacts with CCR5, and not with CXCR4, is a quaternary ammonium derivative, called TAK-779, which also has potent but variable anti-HIV activity (De Clercq et al., Antivir. Chem. Chemoth. 2001, 12 Suppl. 1, 19).

SCH—C(SCH 351125) is another small molecule inhibitor of HIV-1 entry via the CCR5 coreceptor. SCH—C, an oxime-piperidine compound, is a specific CCR5 antagonist as determined in multiple receptor binding and signal transduction assays. This compound specifically inhibits HIV-1 infection mediated by CCR5 in U-87 astroglioma cells but has no effect on infection of CXCR4-expressing cells. (Strizki et al, Proc. Natl. Acad. Sci. USA, 2001, 98(22), 12718 or Tremblay et al., Antimicrobial Agents and Chemotherapy, 2002, 46(5), 1336).

AD101, chemically related to SCH—C, also inhibits the entry of human immunodeficiency virus type 1 (HIV-1) via human CCR5. It has been found that AD101 inhibits HIV-1 entry via rhesus macaque CCR5 while SCH—C does not. Among the eight residues that differ between the human and macaque versions of the coreceptor, only one, methionine-198, accounts for the insensitivity of macaque CCR5 to inhibition by SCH—C. Position 198 is in CCR5 transmembrane (TM) helix 5 and is not located within the previously defined binding site for AD101 and SCH—C, which involves residues in TM helices 1, 2, 3, and 7. Based on studies of amino acid substitutions in CCR5, it has been suggested that the region of CCR5 near residue 198 can influence the conformational state of this receptor. (Billick et al., 2004, J. Virol., 78(8), 4134).

Other known CCR5 inhibitors include, for example, Maraviroc (Dorr et al., Antimicrob Agents Chemother., 2005, 49(11), 4721-32); TAK-220 (Tremblay et al., Antimicrob Agents Chemother., 2005, 49(8), 3483-5; Aplaviroc (Johnson et al., J. Clin Pharmacol., 2006, 46(5), 577-87); TAK-652; vicriviroc (Strizki et al., Antimicrob Agents Chemother., 2005, 49(12), 4911-9); AK602 (Nakata et al., J Virol., 2005, 79(4), 2087-96); SCH-350634 (Tagat et al., J Med Chem., 2001, 44(21), 3343-6); N-(3-[4-(4-fluorobenzoyl)piperidin-1-yl]propyl)-1-methyl-5-oxo-N-phenylpyrrolidine-3-carboxamide (Imamura et al., Chem Pharm Bull (Tokyo), 2004, 52(1), 63-73); N-[3-(4-benzylpiperidin-1-yl)propyl]-N,N′-diphenylurea (Imamura et al., Bioorg Med Chem. 2004, 12(9), 2295-306); and (2S)-2-(3-Chlorophenyl)-1-[N-(methyl)-N-(phenylsulfonyl)amino]-4-[spiro(2,3-dihydro-benzthiophene-3,4′-piperidin-1′-yl)]butane S-oxide (Finke et al., Bioorg Med Chem Lett., 2001, 11(18), 2475-9).

Each of the drugs and drug classes discussed above, when administered alone, can only transiently restrain viral replication. However, some drug combinations can have a profound effect on viremia and disease progression in HIV infection. Still, the high replication rate and rapid turnover of HIV, combined with the frequent incorporation of mutations, leads to treatment failures associated with appearance of drug-resistant variants. Thus, there is continuing need for new methods of treatment and new combinations of therapeutic agents for treatment of HIV infection.

### SUMMARY

According to one embodiment, the present invention provides a pharmaceutical composition comprising:
(A) emtricitabine;
(B) tenofovir disoproxil fumarate;
(C) efavirenz; and
(D) a CCR5 antagonist.

According to a sub-embodiment thereof, there is provided the above pharmaceutical composition, comprising a mixture of amounts of (A), (B), (C) and (D) that is therapeutically effective for treating an HIV infection in a person.

According to another embodiment, the present invention provides a pharmaceutical composition comprising:
(A) lamivudine;
(B) zidovudine;
(C) efavirenz; and
(D) a CCR5 antagonist.

According to a sub-embodiment thereof, there is provided the above pharmaceutical composition, comprising a mixture of amounts of (A), (B), (C) and (D) that is therapeutically effective for treating an HIV infection in a person.

The present invention further provides compositions comprising the above pharmaceutical compositions and a pharmaceutically acceptable carrier.

The present invention further provides methods of treating HIV infection in a patient comprising administering to said patient a therapeutically effective amount of a pharmaceutical composition according to the invention.

The details of one or more embodiments of the invention are set forth in the accompanying description below. Other features, objects, and advantages of the invention will be apparent from the description and from the claims.

### DETAILED DESCRIPTION

The present invention provides, inter alia, methods of treating HIV infection in a person by administering, separately or together, each of the following pharmaceutical agents in amounts that are therapeutically effective for treating HIV infection:
(A) emtricitabine;
(B) tenofovir disoproxil fumarate;
(C) efavirenz; and
(D) at least one CCR5 antagonist.

The present invention further provides methods of treating HIV infection in a person by administering, separately or together, each of the following pharmaceutical agents in amounts that are therapeutically effective for treating HIV infection:
(A) lamivudine;
(B) zidovudine;
(C) efavirenz; and
(D) at least one CCR5 antagonist.

The above combination therapies can be used at any point during treatment, but can also be effective as a first line therapy, that is, for treating HIV infected patients who have not previously undergone antiretroviral therapy.

The pharmaceutical agents of the above combination therapies can be administered together, for example as in a mixture in a pharmaceutical composition, or separately (e.g., simultaneously or sequentially by separate or different routes of administration). In some embodiments, the pharmaceutical agents of the above combination therapies are provided together in a pharmaceutical composition. The pharmaceutical composition can optionally include, in addition to the pharmaceutical agents, at least one pharmaceutically acceptable carrier.

The term “emtricitabine” is meant to include the free base form, pharmaceutically acceptable salt forms, solvates of either the free base or salt forms, hydrates of either the free base or salt forms, crystalline forms (including microcrystalline and nanocrystalline forms), amorphous forms, and isotopically enriched or labeled forms of the compound (4-amino-5-fluoro-1-[2-(hydroxylmethyl)-1,3-oxathiolan-5-yl]-pyrimidin-2-one.

The term “tenofovir disoproxil fumarate” is meant to include the free base form, alternative pharmaceutically acceptable salt forms, solvates of either the free base or salt forms, hydrates of either the free base or salt forms, crystalline forms (including microcrystalline and nanocrystalline forms), amorphous forms, and isotopically enriched or labeled forms of the compound 1-(6-aminopurin-9-yl)propan-2-yloxymethylphosphonic acid.

The term “efavirenz” is meant to include the free base form, pharmaceutically acceptable salt forms, solvates of either the free base or salt forms, hydrates of either the free base or salt forms, crystalline forms (including microcrystalline and nanocrystalline forms), amorphous forms, and isotopically enriched or labeled forms of the compound 8-chloro-5-(2-cyclopropylethynyl)-5-(trifluoromethyl)-4-oxa-2-azabicyclo [4.4.0]deca-7,9,11-trien-3-one.

The term “lamivudine” is meant to include the free base form, pharmaceutically acceptable salt forms, solvates of either the free base or salt forms, hydrates of either the free base or salt forms, crystalline forms (including microcrystalline and nanocrystalline forms), amorphous forms, and isotopically enriched or labeled forms of the compound L-2′,3′-dideoxy-3′-thiacytidine.

The term “zidovudine” is meant to include the free base form, pharmaceutically acceptable salt forms, solvates of either the free base or salt forms, hydrates of either the free base or salt forms, crystalline forms (including microcrystalline and nanocrystalline forms), amorphous forms, and isotopically enriched or labeled forms of the compound 1-[(2R,4S,5S)-4-azido-5-(hydroxymethyl)oxolan-2-yl]-5-methyl-pyrimidine-2,4-dione. The term “zidovudine” is also meant to include prodrugs of zidovudine, for example, fozivudine.

Each of the combination therapies herein includes administration of a CCR5 antagonist. According to some embodiments of the invention, the CCR5 antagonist has a structure according to Formula I:

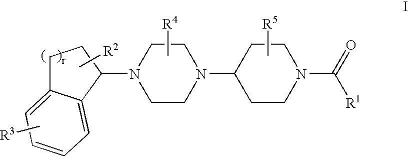

or a pharmaceutically acceptable salt or prodrug thereof, wherein:

R¹ is heteroaryl optionally substituted by one or more R⁶;

R² is H, halo, cyano, nitro, C₁-C₆ alkyl, C₁-C₆ haloalkyl, C₂-C₆ alkenyl, C₂-C₆ alkynyl, aryl, heteroaryl, C₃-C₇ cycloalkyl, heterocycloalkyl, SOR⁷, SO₂R⁷, COR⁸, OR⁹, SR⁹, COOR⁹, NR¹⁰R¹¹ or NR¹⁰COR⁸;

R³ is F, Cl, Br, I, C₁-C₄ haloalkyl, C₁-C₄ haloalkoxy or heteroaryl;

R⁴ is H, C₁-C₆ alkyl, C₂-C₆ alkenyl, C₂-C₆ alkynyl or C₁-C₆ haloalkyl;

R⁵ is H, C₁-C₆ alkyl, C₂-C₆ alkenyl, C₂-C₆ alkynyl or C₁-C₆ haloalkyl;

R⁶ is H, C₁-C₆ alkyl, C₂-C₆ alkenyl, C₂-C₆ alkynyl, C₁-C₆ haloalkyl, C₁-C₆ alkoxy, C₁-C₆ haloalkoxy, amino, (C₁-C₆ alkyl)amino or di(C₁-C₆ alkyl)amino;

R⁷ is H, C₁-C₆ alkyl, C₂-C₆ alkenyl, C₂-C₆ alkynyl, C₁-C₆ haloalkyl, aryl, heteroaryl, C₃-C₇ cycloalkyl, heterocycloalkyl, arylalkyl, heteroarylalkyl, (C₃-C₇ cycloalkyl)alkyl, heterocycloalkylalkyl, or NR¹²R¹³;

R⁸ is H, C₁-C₆ alkyl, C₂-C₆ alkenyl, C₂-C₆ alkynyl, C₁-C₆ haloalkyl, aryl, heteroaryl, C₃-C₇ cycloalkyl, heterocycloalkyl, arylalkyl, heteroarylalkyl, (C₃-C₇ cycloalkyl)alkyl, heterocycloalkylalkyl, or NR¹²R¹³;

R⁹ is H, C₁-C₆ alkyl, C₂-C₆ alkenyl, C₂-C₆ alkynyl, C₁-C₆ haloalkyl, alkoxyalkyl, haloalkoxyalkyl, aryloxyalkyl, heteroaryloxyalkyl, cycloalkyloxyalkyl, heterocycloalkyloxyalkyl, aryl, heteroaryl, C₃-C₇ cycloalkyl, heterocycloalkyl, arylalkyl, heteroarylalkyl; (C₃-C₇ cycloalkyl)alkyl or heterocycloalkylalkyl;

R¹⁰ and R¹¹ are each, independently, H, C₁-C₆ alkyl, C₂-C₆ alkenyl, C₂-C₆ alkynyl, C₁-C₆ haloalkyl, aryl, heteroaryl, C₃-C₇ cycloalkyl, heterocycloalkyl, arylalkyl, heteroarylalkyl; (C₃-C₇ cycloalkyl)alkyl or heterocycloalkylalkyl;

or R¹⁰ and R¹¹ together with the N atom to which they are attached form a 3-, 4-, 5-, 6-, or 7-membered heterocycloalkyl group;

R¹² and R¹³ are each, independently, H, C₁-C₆ alkyl, C₂-C₆ alkenyl, C₂-C₆ alkynyl, C₁-C₆ haloalkyl, aryl, heteroaryl, C₃-C₇ cycloalkyl, heterocycloalkyl, arylalkyl, heteroarylalkyl; (C₃-C₇ cycloalkyl)alkyl or heterocycloalkylalkyl;

or R¹² and R¹³ together with the N atom to which they are attached form a 3-, 4-, 5-, 6-, or 7-membered heterocycloalkyl group; and

r is 1, 2 or 3.

In some embodiments, R¹ is a 5-, 6-, 9- or 10-membered heteroaryl group containing at least one ring-forming N atom, wherein said 5-, 6-, 9- or 10-membered heteroaryl group is optionally substituted by 1, 2, 3 or 4 R⁶ groups.

In some embodiments, R¹ is a 9- or 10-membered heteroaryl group containing at least one ring-forming N atom, wherein said 6-membered heteroaryl group is optionally substituted by 1, 2, 3 or 4 R⁶ groups.

In some embodiments, R¹ is a 6- or 5-membered heteroaryl group containing at least one ring-forming N atom, wherein said 5-membered heteroaryl group is optionally substituted by 1, 2, 3 or 4 R⁶ groups.

In some embodiments, R¹ is a 6-membered heteroaryl group containing at least one ring-forming N atom, wherein said 6-membered heteroaryl group is optionally substituted by 1, 2, 3 or 4 R⁶ groups.

In some embodiments, R¹ is a 5-membered heteroaryl group containing at least one ring-forming N atom, wherein said 5-membered heteroaryl group is optionally substituted by 1, 2, 3 or 4 R⁶ groups.

In some embodiments, R¹ is quinolinyl, isoquinolinyl, naphthyridinyl, indolyl, indazolyl, pyridyl, pyrimidinyl, N-oxopyridyl, N-oxopyrimindinyl, isoxazole, pyrazole, pyrrolyl, imidazolyl, oxazolyl or thiazolyl, each optionally substituted by 1, 2, 3 or 4 R⁶ groups.

In some embodiments, R¹ is quinolinyl, isoquinolinyl, naphthyridinyl, pyridyl, pyrimidinyl, N-oxopyridyl, isoxazole or pyrazole, each optionally substituted by 1, 2, 3 or 4 R⁶ groups.

In some embodiments, R¹ is pyridyl, pyrimidinyl, N-oxopyridyl, N-oxopyrimindinyl, isoxazole, pyrazole, pyrrolyl, imidazolyl, oxazolyl or thiazolyl, each optionally substituted by 1, 2, 3 or 4 R⁶ groups.

In some embodiments, R¹ is pyridyl, pyrimidinyl, N-oxopyridyl, isoxazole or pyrazole, each optionally substituted by 1, 2, 3 or 4 R⁶ groups.

In some embodiments, R¹ is:

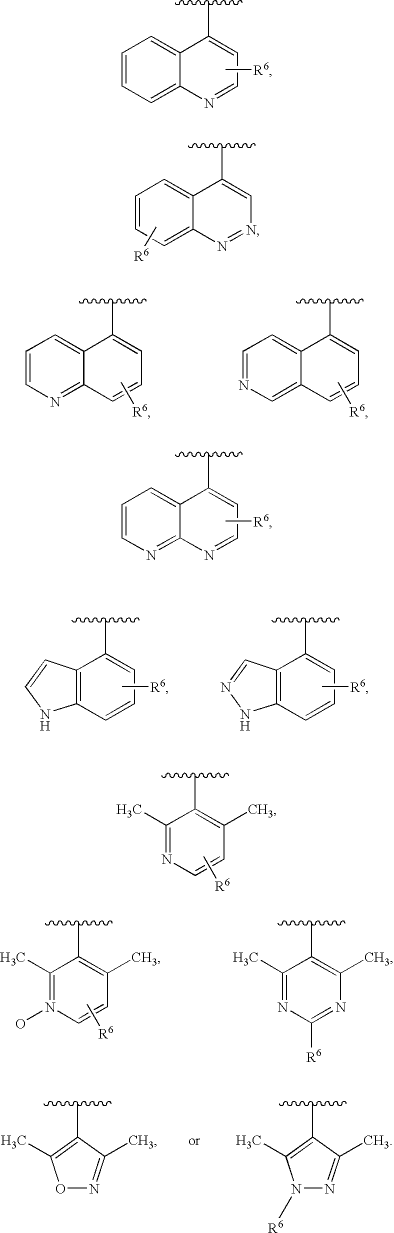

In some embodiments, R¹ is:

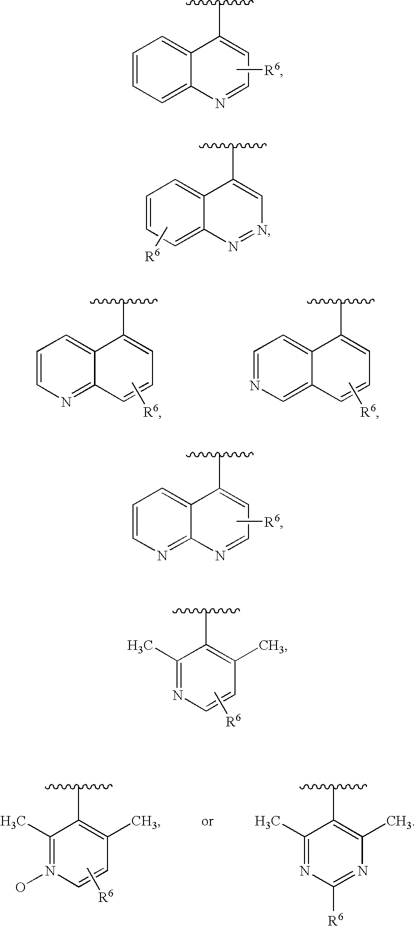

In some embodiments, R¹ is:

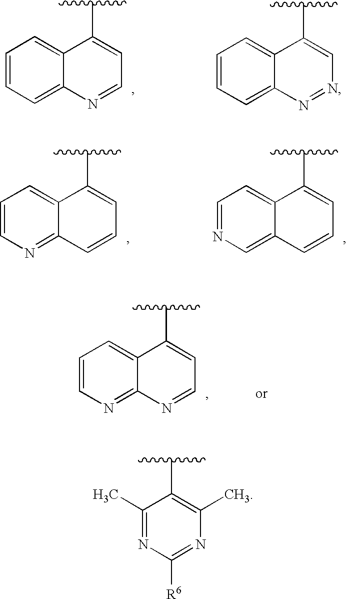

In some embodiments, R¹ is

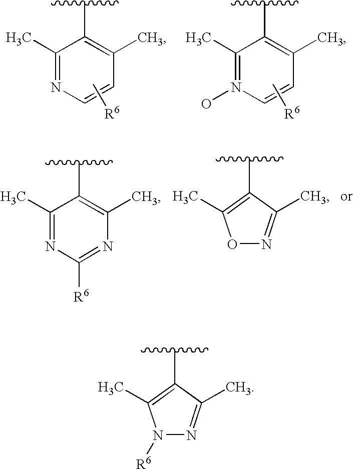

In some embodiments, R¹ is

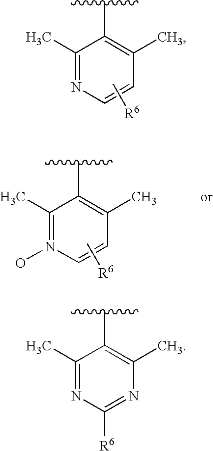

In some embodiments, R¹ is

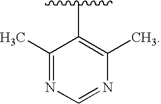

In some embodiments, R² is H, C₁-C₆ alkyl, C₁-C₆ haloalkyl, OR⁹, SR⁹ or NR¹⁰R¹¹.

In some embodiments, R² is H or OR⁹.

In some embodiments, R³ is F, Br, CF₃, or 6- or 5-membered heteroaryl.

In some embodiments, R³ is F, Br, CF₃, OCF₃, thiazolyl, pyrimidinyl, pyridyl.

In some embodiments, R³ is F, Br, or CF₃.

In some embodiments, R⁴ is C₁-C₆ alkyl.

In some embodiments, R⁴ is methyl.

In some embodiments, R⁵ is C₁-C₆ alkyl.

In some embodiments, R⁵ is methyl.

In some embodiments, r is 1.

In some embodiments, r is 2.

In some embodiments, the compound of Formula I in the pharmaceutical composition of the invention is a compound having Formula IIa or IIb:

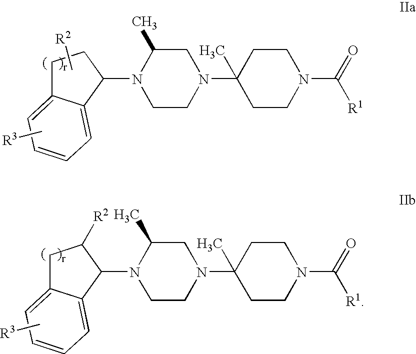

In some embodiments of compounds having Formula IIa or IIb, R¹ is:

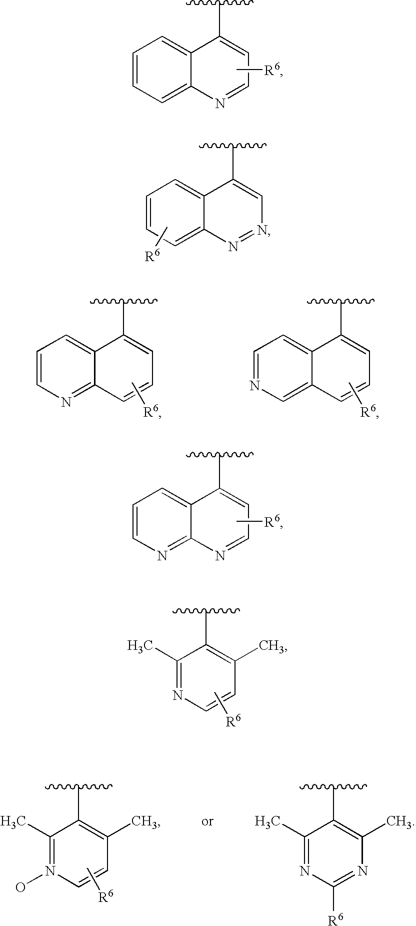

In some embodiments of compounds having Formula IIa or IIb, R¹ is:

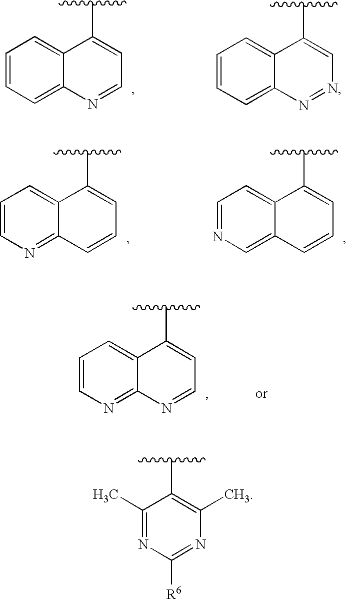

In some embodiments of compounds having Formula IIa or IIb, R¹ is:

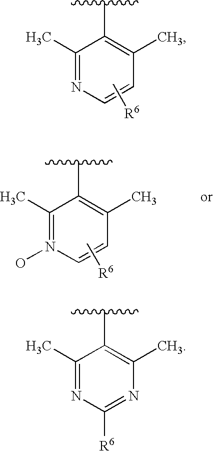

In some embodiments of compounds having Formula IIa or IIb, R¹ is

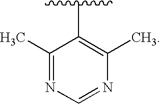

In some embodiments of compounds having Formula IIa or IIb, R² is H, C₁-C₆ alkyl, C₁-C₆ haloalkyl, OR⁹, SR⁹ or NR¹⁰R¹¹.

In some embodiments of compounds having Formula IIa or IIb, R² is H or OR⁹.

In some embodiments of compounds having Formula IIa or IIb, R³ is F, Br, CF₃, 5- or 6-membered heteroaryl.

In some embodiments of compounds having Formula IIa or IIb, R³ is F, Br, or CF₃.

According to an embodiment of the invention, the CCR5 component of the pharmaceutical composition is a compound of Formula I selected from:
5-({4-[(3S)-4-(5-bromo-2,3-dihydro-1H-inden-1-yl)-3-methylpiperazin-1-yl]-4-methylpiperidin-1-yl}carbonyl)-4,6-dimethylpyrimidine;
5-({4-[(3S)-4-(5-fluoro-2,3-dihydro-1H-inden-1-yl)-3-methylpiperazin-1-yl]-4-methylpiperidin-1-yl}carbonyl)-4,6-dimethylpyrimidine;
5-({4-[(3S)-4-(6-bromo-2,3-dihydro-1H-inden-1-yl)-3-methylpiperazin-1-yl]-4-methylpiperidin-1-yl}carbonyl)-4,6-dimethylpyrimidine;
5-({4-[(3S)-4-(6-fluoro-2,3-dihydro-1H-inden-1-yl)-3-methylpiperazin-1-yl]-4-methylpiperidin-1-yl}carbonyl)-4,6-dimethylpyrimidine;
5-({4-[(3S)-4-(6-bromo-1,2,3,4-tetrahydronaphthalen-1-yl)-3-methylpiperazin-1-yl]-4-methylpiperidin-1-yl}carbonyl)-4,6-dimethylpyrimidine;
5-({4-[(3S)-4-(7-bromo-1,2,3,4-tetrahydronaphthalen-1-yl)-3-methylpiperazin-1-yl]-4-methylpiperidin-1-yl}carbonyl)-4,6-dimethylpyrimidine;
4,6-dimethyl-5-[(4-methyl-4-{(3S)-3-methyl-4-[6-(trifluoromethyl)-2,3-dihydro-1H-inden-1-yl]piperazin-1-yl}piperidin-1-yl)carbonyl]pyrimidine;
4,6-dimethyl-5-[(4-methyl-4-{(3S)-3-methyl-4-[5-(trifluoromethyl)-2,3-dihydro-1H-inden-1-yl]piperazin-1-yl}piperidin-1-yl)carbonyl]pyrimidine;
1-((2S)-4-{1-[(4,6-dimethylpyrimidin-5-yl)carbonyl]-4-methylpiperidin-4-yl}-2-methylpiperazin-1-yl)-5-(trifluoromethyl)indan-2-ol;
5-[(4-{(3S)-4-[2-methoxy-5-(trifluoromethyl)-2,3-dihydro-1H-inden-1-yl]-3-methylpiperazin-1-yl}-4-methylpiperidin-1-yl)carbonyl]-4,6-dimethylpyrimidine;
5-[(4-(3S)-4-[(1R,2R)-2-ethoxy-5-(trifluoromethyl)-2,3-dihydro-1H-inden-1-yl]-3-methylpiperazin-1-yl-4-methylpiperidin-1-yl)carbonyl]-4,6-dimethylpyrimidine;
5-[(4-{(3S)-4-[(1R,2R)-2-(2-methoxyethoxy)-5-(trifluoromethyl)-2,3-dihydro-1H-inden-1-yl]-3-methylpiperazin-1-yl}-4-methylpiperidin-1-yl)carbonyl]-4,6-dimethylpyrimidine;
4-[(4-{(3S)-4-[(1S,2R)-2-ethoxy-5-(trifluoromethyl)-2,3-dihydro-1H-inden-1-yl]-3-methylpiperazin-1-yl}-4-methylpiperidin-1-yl)carbonyl]cinnoline;
4-[(4-{(3S)-4-[(1R,2R)-2-ethoxy-5-(trifluoromethyl)-2,3-dihydro-1H-inden-1-yl]-3-methylpiperazin-1-yl}-4-methylpiperidin-1-yl)carbonyl]quinoline;
5-[(4-{(3S)-4-[(1R,2R)-2-ethoxy-5-(trifluoromethyl)-2,3-dihydro-1H-inden-1-yl]-3-methylpiperazin-1-yl}-4-methylpiperidin-1-yl)carbonyl]quinoline;
4-[(4-{(3S)-4-[(1R,2R)-2-ethoxy-5-(trifluoromethyl)-2,3-dihydro-1H-inden-1-yl]-3-methylpiperazin-1-yl}-4-methylpiperidin-1-yl)carbonyl]-1,8-naphthyridine;
5-[(4-{(3S)-4-[(1R,2R)-2-ethoxy-5-(trifluoromethyl)-2,3-dihydro-1H-inden-1-yl]-3-methylpiperazin-1-yl}-4-methylpiperidin-1-yl)carbonyl]isoquinoline;
5-[(4-{(3S)-4-[(1R,2R)-5-bromo-2-ethoxy-2,3-dihydro-1H-inden-1-yl]-3-methylpiperazin-1-yl}-4-methylpiperidin-1-yl)carbonyl]-4,6-dimethylpyrimidine;
4-[(4-{(3S)-4-[(1R,2R)-5-bromo-2-ethoxy-2,3-dihydro-1H-inden-1-yl]-3-methylpiperazin-1-yl}-4-methylpiperidin-1-yl)carbonyl]cinnoline;
4-[(4-{(3S)-4-[(1R,2R)-5-bromo-2-ethoxy-2,3-dihydro-1H-inden-1-yl]-3-methylpiperazin-1-yl}-4-methylpiperidin-1-yl)carbonyl]-1,8-naphthyridine;
5-[(4-{(3S)-4-[(1R,2R)-5-bromo-2-(pyridin-2-yloxy)-2,3-dihydro-1H-inden-1-yl]-3-methylpiperazin-1-yl}-4-methylpiperidin-1-yl)carbonyl]-4,6-dimethylpyrimidine;
5-[(4-{(3S)-4-[(1R,2R)-2-ethoxy-5-(1,3-thiazol-2-yl)-2,3-dihydro-1H-inden-1-yl]-3-methylpiperazin-1-yl}-4-methylpiperidin-1-yl)carbonyl]-4,6-dimethylpyrimidine;
5-[(4-{(3S)-4-[(1R,2R)-2-ethoxy-5-pyridin-2-yl-2,3-dihydro-1H-inden-1-yl]-3-methylpiperazin-1-yl}-4-methylpiperidin-1-yl)carbonyl]-4,6-dimethylpyrimidine;
5-[(4-{(3S)-4-[3-methoxy-5-(trifluoromethyl)-2,3-dihydro-1H-inden-1-yl]-3-methylpiperazin-1-yl}-4-methylpiperidin-1-yl)carbonyl]-4,6-dimethylpyrimidine;

and pharmaceutically acceptable salts thereof.

The above compounds of Formula I are described and can be prepared according to the methods described in U.S. Pat. App. Pub. No. 2005/0261310, which is incorporated herein by reference in its entirety.

Other suitable CCR5 antagonists are described in, for example, U.S. Pat. Nos. 6,936,602; 6,855,724; 6,787,650; 6,689,783; 6,689,765; 6,635,646; 6,602,885; 6,562,978; 6,515,027; 6,506,790; 6,399,656; 6,391,865; 6,387,930; 6,376,536; 6,268,354; 6,242,459; 6,235,771; 6,172,061; 6,096,780; and U.S. Pat. App. Pub. Nos. 2006/0178359; 2006/0160864; 2006/0122166; 2006/0105964; 2006/0100197; 2006/0058284; 2006/0052595; 2006/0047116; 2006/0025441; 2006/0014767; 2006/0004047; 2005/0131011; 2005/0107424; 2004/0259876; 2004/0235823; 2004/0142920; 2004/0072818; 2004/0067961; 2004/0053936; 2004/0038982; 2004/0014742; 2003/0114443; 2003/0087912; 2003/0078189; 2003/0069252; and 2003/0004185, each of which is incorporated herein by reference in its entirety. Further suitable CCR5 antagonists are selected from:
4-[[6-amino-5-bromo-2-[(4-cyanophenyl)amino]-4-pyrimidinyl]oxy]-3,5-dimethylbenzonitrile (TMC-125);
N,N-dimethyl-N-[4-[[[2-(4-methylphenyl)-6,7-dihydro-5H-benzocyclohepten-8-yl]-carbonyl]amino]benzyl]-tetrahydro-2H-pyran-4-aminium chloride (TAK-779);
(4,4-difluoro-N-[(1S)-3-[exo-3-(3-isopropyl-5-methyl-4H-1,2,4-triazol-4-yl)-8-azabicyclo [3.2.1]oct-8-yl]-1-phenylpropyl]cyclohexanecarboxamide) (Maraviroc);
1-acetyl-N-(3-(4-(4-carbamoylbenzyl)piperidin-1-yl)propyl)-N-(3-chloro-4-methylphenyl)piperidine-4-carboxamide (TAK-220);
4-[(Z)-(4-bromophenyl)-(ethoxyimino)methyl]-1′-[(2,4-dimethyl-3-pyridinyl)-carbonyl]-4′-methyl-1,4′-bipiperidine N-oxide (SCH-351125);
4-{[4-({(3R)-1-Butyl-3-[(R)-cyclohexyl(hydroxy)methyl]-2,5-dioxo-1,4,9-triazaspiro-[5.5]undec-9-yl}methyl)phenyl]oxy}benzoic acid (Aplaviroc);
(S)-8-[4-(2-butoxyethoxy)phenyl]-1-isobutyl-N-(4-{[(1-propyl-1H-imidazol-5-yl)-methyl]sulfinyl}phenyl)-1,2,3,4-tetrahydro-1-benzazocine-5-carboxamide monomethane-sulfonate (TAK-652);
1-[(4,6-dimethyl-5-pyrimidinyl)carbonyl]-4-[4-[2-methoxy-1(R)-4-(trifluoromethyl)-phenyl]ethyl-3 (S)-methyl-1-piperazinyl]-4-methylpiperidine (vicriviroc);
4-(4-(((R)-1-butyl-3-((R)-cyclohexyl(hydroxy)methyl)-2,5-dioxo-1,4,9-triazaspiro-[5.5]undecan-9-yl)methyl)phenoxy)benzoic acid hydrochloride (AK602);
1-[(2,4-dimethyl-3-pyridinyl)carbonyl]-4-methyl-4-[3 (S)-methyl-4-[1(S)-[4-(trifluoromethyl)phenyl]ethyl]-1-piperazinyl]-piperidine N1-oxide (Sch-350634);
N-(3-[4-(4-fluorobenzoyl)piperidin-1-yl]propyl)-1-methyl-5-oxo-N-phenyl-pyrrolidine-3-carboxamide;
N-[3-(4-benzylpiperidin-1-yl)propyl]-N,N′-diphenylurea;
(2S)-2-(3-Chlorophenyl)-1-[N-(methyl)-N-(phenylsulfonyl)amino]-4-[spiro(2,3-dihydrobenzthiophene-3,4′-piperidin-1′-yl)]butane S-oxide; and
(4,6-dimethylpyrimidin-5-yl)(4-methyl-4-((S)-3-methyl-4-((S)-1-(4-(trifluoromethyl)-phenyl)ethyl)piperazin-1-yl)piperidin-1-yl)methanone (AD-101), as well as salt forms and free base forms thereof.

The combinations of compounds in the pharmaceutical compositions referred to above may conveniently be presented for use in the form of a pharmaceutical formulation and thus pharmaceutical formulations comprising a combination of compounds as defined above together with a pharmaceutically acceptable carrier therefore comprise a further aspect of the invention.

The individual components of such combinations may be administered either sequentially or simultaneously in separate or combined pharmaceutical formulations.

It is appreciated that certain features of the invention, which are, for clarity, described in the context of separate embodiments, can also be provided in combination in a single embodiment. Conversely, various features of the invention which are, for brevity, described in the context of a single embodiment, may also be provided separately or in any suitable subcombination.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl) and the like. An alkyl group can contain from 1 to about 20, from 2 to about 20, from 1 to about 10, from 1 to about 8, from 1 to about 6, from 1 to about 4, or from 1 to about 3 carbon atoms.

As used herein, “alkenyl” refers to an alkyl group having one or more double carbon-carbon bonds. Example alkenyl groups include ethenyl, propenyl, butenyl, pentenyl, hexenyl, butadienyl, pentadienyl, hexadienyl, and the like.

As used herein, “alkynyl” refers to an alkyl group having one or more triple carbon-carbon bonds. Example alkynyl groups include ethynyl, propynyl, butynyl, pentynyl, and the like.

As used herein, “haloalkyl” refers to an alkyl group having one or more halogen substituents. Example haloalkyl groups include CF₃, C₂F₅, CHF₂, CCl₃, CHCl₂, C₂Cl₅, and the like. An alkyl group in which all of the hydrogen atoms are replaced with halogen atoms can be referred to as “perhaloalkyl.” Example perhaloalkyl groups include CF₃ and C₂F₅.

As used herein, “aryl” refers to monocyclic or polycyclic aromatic hydrocarbons such as, for example, phenyl, naphthyl, anthracenyl, phenanthrenyl, indanyl, indenyl, and the like. In some embodiments, aryl groups have from 6 to about 18 carbon atoms.

As used herein, “cycloalkyl” refers to non-aromatic cyclic hydrocarbons, including cyclized alkyl, alkenyl, and alkynyl groups. Cycloalkyl groups can include bi- or poly-cyclic ring systems and can optionally contain unsaturations. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (i.e., having a bond in common with) to the cycloalkyl ring, for example, benzo derivatives of cyclopentane (indanyl), cyclohexane (tetrahydronaphthyl), and the like. Cycloalkyl groups can have from about 3 to about 20, 3 to about 12, or 3 to about 7 carbon atoms.

As used herein, “heteroaryl” groups are monocyclic and polycyclic aromatic hydrocarbons that have at least one heteroatom ring member such as sulfur, oxygen, or nitrogen. Heteroaryl groups include, without limitation, pyridyl, N-oxopyridyl, pyrimidinyl, N-oxopyrimidinyl, pyrazinyl, pyridazinyl, triazinyl, naphthyridinyl, furyl, quinolyl, iso-quinolyl, thienyl, imidazolyl, thiazolyl, indolyl, pyrrolyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, 2,3-dihydrobenzofuranyl, 2,3-dihydrobenzothienyl, 2,3-dihydrobenzothienyl-5-oxide, 2,3-dihydrobenzothienyl-5-dioxide, and the like. In some embodiments, heteroaryl groups can have from 1 to about 20 carbon atoms, and in further embodiments from about 3 to about 20 carbon atoms. In some embodiments, heteroaryl groups have 1 to about 4, 1 to about 3, or 1 to 2 heteroatoms. In some embodiments, the heteroaryl group has 5 to 50, 5 to 20, 5 to 14 or 5 to 7 ring members. In some embodiments, the heteroaryl group is a 5-, 6-, 9-, or 10-membered group. In some embodiments, the heteroaryl group contains at least one ring-forming N atom.

As used herein, “heterocycloalkyl” refers to a cyclized, non-aromatic hydrocarbon including cyclized alkyl, alkenyl, and alkynyl groups where one or more of the ring-forming carbon atoms is replaced by a heteroatom such as an O, N, or S atom. Example heterocycloalkyl groups include piperidinyl, pyrrolidinyl, morpholino, tetrahydrofuranyl, and the like. Also included in the definition of heterocycloalkyl are moieties that have one or more aromatic rings fused (i.e., having a bond in common with) to the non-aromatic heterocyclic ring, for example phthalimidyl, naphthalimidyl pyromellitic diimidyl, phthalanyl, and benzo derivatives of saturated heterocycles such as indolene and isoindolene groups. In some embodiments, the heterocycloalkyl group has 3 to 20, 3 to 14 or 3 to 7 ring members.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like. “Haloalkoxy” refers to an —O-haloalkyl group.

As used here, “arylalkyl” refers to an alkyl group substituted by at least one aryl group. An example arylalkyl group is benzyl.

As used herein, “cycloalkylalkyl” refers to an alkyl group substituted by at least one cycloalkyl group.

As used herein, “heteroarylalkyl” refers to an alkyl group substituted by at least one heteroaryl group.

As used herein, “heterocycloalkylalkyl” refers to an alkyl group substituted by at least one heterocycloalkyl group.

As used herein, “aryloxy” refers to —O-aryl.

As used herein, “heteroaryloxy” refers to —O-heteroaryl.

As used herein, “cycloalkyloxy” refers to —O-cycloalkyl.

As used herein, “heterocycloalkyloxy” refers to —O-heterocycloalkyl.

As used herein, “alkoxyalkyl” refers to an alkyl group substituted by at least one alkoxy group. Example alkoxyalkyl groups include methoxymethyl, methoxyethyl, methoxypropyl and the like.

As used herein, “haloalkoxyalkyl” refers to an alkyl group substituted by at least one haloalkoxy group.

As used herein, “arylalkoxyalkyl” refers to an alkyl group substituted by at least one aryloxy group.

As used herein, “cycloalkyloxyalkyl” refers to an alkyl group substituted by at least one cycloalkyloxy group.

As used herein, “heteroaryloxyalkyl” refers to an alkyl group substituted by at least one heteroaryloxy group.

As used herein, “heterocycloalkloxyalkyl” refers to an alkyl group substituted by at least one heterocycloalkyloxy group.

As used herein, the term “amino” refers to NH₂. Similarly, the term “alkylamino” refers to an amino group substituted by an alkyl group, and the term “dialkylamino” refers to an amino group substituted by two alkyl groups.

As used herein, “substituted” indicates that at least one hydrogen atom of a chemical group is replaced by a non-hydrogen moiety. When a chemical group herein is “substituted” it may have up to the full valance of substitution, provided the resulting compound is a stable compound or stable structure; for example, a methyl group may be substituted by 1, 2, or 3 substituents, a methylene group may be substituted by 1 or 2 substituents, a phenyl group may be substituted by 1, 2, 3, 4, or 5 substituents, and the like.

The CCR5 antagonist compounds, e.g., compounds of Formulae I, IIa and IIb that are described herein can be asymmetric (e.g., having one or more stereocenters). All stereoisomers, such as enantiomers and diastereomers, are intended unless otherwise indicated. Compounds of Formulae I, IIa and IIb, present in compositions according to the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of Formulae I, IIa and IIb present in compositions of the present invention are described and may be isolated as a mixture of isomers or as separated isomeric forms.

Resolution of racemic mixtures of compounds can be carried out by any of numerous methods known in the art. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of Formulae I, IIa and IIb present in pharmaceutical compositions of the invention can also include tautomeric forms, such as keto-enol tautomers. Tautomeric forms can be in equilibrium or sterically locked into one form by appropriate substitution.

Compounds of Formulae I, IIa and IIb present in pharmaceutical compositions of the invention also include hydrates and solvates.

Compounds of Formulae I, IIa and IIb, present in pharmaceutical compositions of the invention, can also include all isotopes of atoms occurring in the intermediates or final compounds. Isotopes include those atoms having the same atomic number but different mass numbers. For example, isotopes of hydrogen include tritium and deuterium.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes compositions comprising pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of compounds used in compositions of the present invention include the conventional non-toxic salts or the quaternary ammonium salts of the parent compound formed, for example, from non-toxic inorganic or organic acids. The pharmaceutically acceptable salts of compounds used in compositions of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The present invention also includes compositions wherein one or more of the component compounds are present as prodrugs of the compounds described herein. As used herein, “prodrugs” refer to any covalently bonded carriers which release the active parent drug when administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Examples of prodrugs include, but are not limited to, acetate, formate and benzoate derivatives of alcohol and amine functional groups in the compounds of the invention. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Methods of Treatment

The present invention pertains to methods for treating an HIV infection in a person, comprising administering to the person, separately or together, therapeutically effective amounts of: (A) emtricitabine; (B) tenofovir disoproxil fumarate; (C) efavirenz; and (D) a CCR5 antagonist.

According to further embodiments of the invention, there are provided methods for treating an HIV infection in a person, comprising administering to the person, separately or together, therapeutically effective amounts of: (A) lamivudine; (B) zidovudine disoproxil fumarate; (C) efavirenz; and (D) a CCR5 antagonist.

According to further embodiments, there are provided methods for treating an HIV infection in a person, comprising administering to the person a therapeutically effective amount of a pharmaceutical composition comprising (A) emtricitabine; (B) tenofovir disoproxil fumarate; (C) efavirenz; and (D) a CCR5 antagonist.

According to further embodiments, there are provided methods for treating an HIV infection in a person, comprising administering to the person a therapeutically effective amount of a pharmaceutical composition comprising (A) lamivudine; (B) zidovudine disoproxil fumarate; (C) efavirenz; and (D) a CCR5 antagonist.

In some embodiments, the compositions according to the invention are administered in a solid dosage form. According to an embodiment, the solid dosage form administered according to the method of the invention is administered orally. According to another embodiment, the methods according to the invention are carried out according to a dosage regimen wherein a solid dosage form is administered no more than once per day, no more than once per 48 hour period, or no more than once per week. According to some embodiments, the solid dosage form is administered once per week.

According to an embodiment of methods of the invention, the compositions according to the invention are administered as a first line therapy for treatment of HIV, i.e., the method comprises treating an HIV infection in a person who has not previously received antiretroviral therapy.

The lamivudine, zidovudine, emtricitabine and efavirenz in the compositions and methods of the invention can bind to the viral reverse transcriptase enzyme and inhibit HIV replication by inhibiting the action of reverse transcriptase.

CCR5 antagonist compounds of Formula I in the compositions and methods of the invention can inhibit activity of CCR5 and optionally modulate activity of one or more other chemokine receptors. The term “modulate” is meant to refer to an ability to increase or decrease activity of a receptor. Accordingly, CCR5 antagonist compounds of Formulae I, IIa and IIb can be used in methods of modulating a chemokine receptor by contacting the receptor with any one or more of the compounds or compositions described herein that contain the compounds.

The CCR5 antagonist compounds can bind to a chemokine receptor in such a way to block or inhibit binding of endogenous and other chemokine receptor ligands. In some embodiments, the CCR5 antagonist compounds can block or inhibit binding of exogenous ligands including viral proteins involved in viral entry into cells expressing the chemokine receptor. Accordingly, the CCR5 antagonist compounds in compositions and methods of the invention can block viral entry and inhibit viral infection. In some embodiments, CCR5 antagonist compounds in compositions of the invention can inhibit HIV infection by, for example, blocking interaction of a CCR5 receptor with HIV glycoprotein120 (gp120).

The CCR5 antagonist compounds present in compositions and methods of the invention can be selective. By “selective” is meant that the compound binds to or inhibits a CCR5 receptor with greater affinity or potency, respectively, compared to at least one other chemokine receptor.

The CCR5 antagonist compounds can be selective binders of CCR5, meaning that the compounds can bind to CCR5 with greater affinity than for another chemokine receptor such as at least one of CCR1, CCR2, CCR3, CCR4, CCR6, CCR7 and CCR8. In some embodiments, the CCR5 antagonist compounds have binding selectivity for CCR5 over CCR2. In some embodiments, the CCR5 antagonist compounds have binding selectivity for CCR5 over CCR1. In some embodiments, the CCR5 antagonist compounds have binding selectivity for CCR5 over any other CCR. Selectivity can be at least about 10-fold, at least about 20-fold, at least about 50-fold, at least about 100-fold, at least about 200-fold, at least about 500-fold or at least about 1000-fold. In some embodiments, the CCR5 antagonist compounds have binding affinity for CCR5 that is at least about 10-fold, at least about 20-fold, at least about 50-fold, at least about 100-fold, at least about 200-fold, at least about 500-fold or at least about 1000-fold greater than binding affinity for CCR1, CCR2 or any other chemokine receptor. Binding affinity can be measured according to routine methods in the art, such as according to the assays provided herein.

CCR5 antagonist compounds present in compositions of the invention can be selective inhibitors of CCR5, meaning that the compounds can inhibit activity of CCR5 more potently than for at least one other chemokine receptors such as, for example, CCR1, CCR2, CCR3, CCR4, CCR6, CCR7 and CCR8. In some embodiments, the CCR5 antagonist compounds have inhibition selectivity for CCR5 over CCR2. In some embodiments, the CCR5 antagonist compounds have inhibition selectivity for CCR5 over CCR1. In some embodiments, the CCR5 antagonist compounds have inhibition selectivity for CCR5 over any other CCR. Selectivity can be at least about 10-fold, at least about 20-fold, at least about 50-fold, at least about 100-fold, at least about 200-fold, at least about 500-fold or at least about 1000-fold. In some embodiments, the compounds have inhibition affinity for CCR5 that is at least about 10-fold, at least about 20-fold, at least about 50-fold, at least about 100-fold, at least about 200-fold, at least about 500-fold or at least about 1000-fold greater than binding affinity for CCR1, CCR2 or any other chemokine receptor. Inhibitor potency can be measured according to routine methods in the art, such as according to the assays provided herein.

As used herein, the term “contacting” refers to the bringing together of indicated moieties in an in vitro system or an in vivo system. For example, “contacting” a CCR5 receptor, with a composition of the invention includes the administration of the composition, by administering the compounds in the composition together or separately, to a person or patient, e.g., a human, having a chemokine receptor. Likewise, “contacting” a viral reverse transcriptase enzyme with a composition of the invention includes administration of the compounds in the composition together or separately, to a person infected with HIV. The term “contacting” also refers, for example, introducing a composition of the invention into a sample containing a cellular or purified preparation containing the chemokine receptor.

As used herein, the term “person” or “patient,” used interchangeably, refers to a human, or to any animal, including mammals, such as mice, rats, other rodents, rabbits, dogs, cats, swine, cattle, sheep, horses, or primates. In some embodiments, the person is infected with HIV and has not previously undergone antiretroviral therapy.

As used herein, “antiretroviral therapy” is meant to have the meaning commonly known in the art which is a treatment for AIDS involving administering of at least one antiretroviral agent (or, commonly, a cocktail of antiretrovirals) such as nucleoside reverse transcriptase inhibitor (e.g., zidovudine (AZT, lamivudine (3TC) and abacavir), non-nucleoside reverse transcriptase inhibitor (e.g., nevirapine and efavirenz), and protease inhibitor (e.g., indinavir, ritonavir and lopinavir).

As used herein, the phrase “therapeutically effective amount” refers to the amount of a pharmaceutical composition of the invention, or of an individual compound in a pharmaceutical composition of the invention, that elicits the biological or medicinal response in a tissue, system, animal, individual or human that is being sought by a researcher, veterinarian, medical doctor or other clinician, which includes one or more of the following:

(1) inhibiting HIV in a person who is experiencing or displaying the pathology or symptomotology related to HIV infection (i.e., arresting further development of the pathology and/or symptomotology) such as stabilizing viral load;

(2) ameliorating the disease; for example, ameliorating a HIV-related disease, condition or disorder in a person who is experiencing or displaying the pathology or symptomotology of the disease, condition or disorder (i.e., reversing the pathology and/or symptomotology) such as lowering viral load in the case of a viral infection and

(3) preventing HIV infection or a disease associated with HIV infection; for example, treatment with compositions of the invention may be carried out to substantially reduce the risk of HIV infection following an actual or potential exposure to HIV (e.g., a needle-stick injury involving blood or body fluids from an individual known to be infected with HIV) when the patient does not yet experience or display the pathology or symptomotology of HIV infection or of a HIV-related disease;

(4) preventing mother-to-child transmission of HIV during pregnancy, labor and delivery or cesarean section procedure.

Additional Pharmaceutical Agents

One or more additional pharmaceutical agents such as, for example, anti-viral agents, antibodies, anti-inflammatory agents, analgesics, and/or immunosuppressants can be used in combination with the compositions of the present invention for treatment of HIV. The agents can be combined with the present compositions in a single dosage form, or the agents can be administered simultaneously or sequentially as separate dosage forms.

Suitable antiviral agents contemplated for use in combination with the compositions of the present invention can comprise additional nucleoside and nucleotide reverse transcriptase inhibitors (NRTIs and NtTRIs) or non-nucleoside reverse transcriptase inhibitors (NNRTIs), and also protease inhibitors, integrase inhibitors, maturation inhibitors, other CCR5 antagonists, fusion inhibitors and other antiviral drugs.

Additional suitable NRTIs include, for example, GS7340 (Gilead Sciences), GS9148 (Gilead Sciences), elvucitabine, didanosine (ddI); zalcitabine (ddC); stavudine (d4T); abacavir (1592U89); adefovir dipivoxil [bis(POM)-PMEA]; lobucavir (BMS-180194); BCH-10652; beta-L-FD4 (also called beta-L-D4C and named beta-L-2′,3′-dicleoxy-5-fluoro-cytidene); DAPD, ((−)-beta-D-2,6,-diamino-purine dioxolane); and lodenosine (FddA).

Additional suitable NNRTIs include, for example, TMC278 (Tibotec Pharmaceuticals), nevirapine (BI-RG-587); delaviradine (BHAP, U-90152); PNU-142721; AG-1549; MKC-442 (1-(ethoxy-methyl)-5-(1-methylethyl)-6-(phenylmethyl)-(2,4(1H,3H)-pyrimidinedione); TMC125; and (+)-calanolide A (NSC-675451) and B (NSC-661122).

Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; atazanavir (BMS-2322623); lopinavir (ABT-378); darunavir (TMC114); brecanavir; tipranavir; and AG-1 549.

Typical suitable integrase inhibitors include MK0518 and GS9137.

Typical suitable maturation inhibitors include berivamat (PA457).

Typical suitable CCR5 antagonists include maraviroc and vicriviroc.

Typical suitable fusion inhibitors include T-20, TR1-1144 and TR1-999.

Other antiviral agents include hydroxyurea, ribavirin, IL-2, IL-12, pentafuside and Yissum Project No. 11607.

In some embodiments, anti-inflammatory or analgesic agents contemplated for use in combination with the compositions of the present invention can comprise, for example, an opiate agonist, a lipoxygenase inhibitor such as an inhibitor of 5-lipoxygenase, a cyclooxygenase inhibitor such as a cyclooxygenase-2 inhibitor, an interleukin inhibitor such as an interleukin-I inhibitor, an NNMA antagonist, an inhibitor of nitric oxide or an inhibitor of the synthesis of nitric oxide, a non-steroidal antiinflammatory agent, or a cytokine-suppressing antiinflammatory agent, for example, such as acetaminophen, aspirin, codeine, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds can be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedfine, or levo-desoxyephedrine; an antitussive such as codeine, hydrocodone, caramiphen, carbetapentane, or dextramethorphan; a diuretic; and a sedating or non-sedating antihistamine.

In some embodiments, pharmaceutical agents contemplated for use in combination with the compositions of the present invention can comprise (a) VLA-4 antagonists such as those described in U.S. Pat. No. 5,510,332, W095/15973, W096/01644, W096/06108, W096/20216, W096/229661, W096/31206, W096/4078, W097/030941, W097/022897 WO 98/426567 W098/53814, W098/53817, W098/538185, W098/54207, and W098/58902; (b) steroids such as beclornethasone, methylprednisolone, betamethasone, prednisone, dexamethasone, and hydrocortisone; (c) immunosuppressants such as cyclosporin, tacrolimus, rapamycin and other FK506 type immunosuppressants; (d) antihistamines (HI-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, asternizole, terfenadine, loratadine, cetirizine, fexofenadine, desearboethoxyloratadine, and the like; (e) non-steroidal anti-asthmatics such as terbutaline, metaproterenol, fenoterol, isoethaiine, albuterol, bitolterol, pirbuterol, theophylline, cromolyn sodium, atropine, ipratropium bromide, leukotriene antagonists (e.g., zafirlukast, montelukast, pranlukast, iralukast, pobilukast, SKB-106,203), leukotriene biosynthesis inhibitors (e.g., zileuton, BAY-1005); (f) nonsteroidal antiinflammatory agents (NSAIDs) such as propionic acid derivatives (e.g., alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (e.g., indomethacin, acernetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenarnic acid derivatives (flufenamic acid, meclofenamic acid, rnefenamic acid, niflumic acid and tolfenamic acid), biphenylearboxylic acid derivatives (diflunisal and flufenisal), oxicarns (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors; (h) inhibitors of phosphodiesterase type IV (PDE-IV); (i) other antagonists of the chemokine receptors, especially CXCR-4, CCRI, CCR2, CCR3 and CCR5; (j) cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (k) anti-diabetic agents such as insulin, sulfonylureas, biguanides (metformin), U.-glucosidase inhibitors (acarbose) and orlitazones (troglitazone and pioglitazone); (1) preparations of interferon beta (interferon beta-lo., interferon beta-1 P); (m) other compounds such as aminosalicylic acids, antimetabolites such as azathioprine and 6-mercaptopurine, and cytotoxic cancer chemotherapeutic agents. The weight ratio of the compound of the compound of the present invention to the second active ingredient may be varied and will depend upon the effective dose of each ingredient.

In some embodiments, additional pharmaceutical agents include any of the above referenced agents or other agents except NRTIs. In further embodiments, additional pharmaceutical agents include any of the above reference agents or other agents except elvucitabine and its salts, hydrates, and solvates.

Pharmaceutical Formulations and Dosage Forms

When employed as pharmaceuticals, the compositions of the invention can be administered in the form of single pharmaceutical compositions in combination with one or more pharmaceutically acceptable carriers. Accordingly, the invention also includes pharmaceutical compositions which contain the active ingredients, i.e., either (A) emtricitabine; (B) tenofovir disoproxil fumarate; (C) efavirenz; and (D) a CCR5 antagonist; or (A) lamivudine; (B) zidovudine; (C) efavirenz; and (D) a CCR5 antagonist, in combination with one or more pharmaceutically acceptable carriers. According to an embodiment, the composition according to the invention comprises solid dosage form. According to a sub-embodiment thereof the solid dosage form is suitable for oral administration. According to another sub-embodiment thereof, the solid dosage form is suitable for once-a-day dosing.

These pharmaceutical compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

In making the compositions of the invention, the active ingredients are typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, capsules, powders, lozenges, sachets, cachets, elixirs, suspensions, emulsions, solutions, syrups, aerosols (as a solid or in a liquid medium), ointments containing, for example, up to 10% by weight of the active compounds, soft and hard gelatin capsules, suppositories, sterile injectable solutions, and sterile packaged powders.

In preparing a formulation, the active compounds can be milled, separately or together, to provide the appropriate particle size prior to combining with the other ingredients. If an active compound is substantially insoluble, it can be milled to a particle size of less than 200 mesh. If an active compound is substantially water soluble, the particle size can be adjusted by milling to provide a substantially uniform distribution in the formulation, e.g. about 40 mesh.

The active compounds may be milled using known milling procedures such as wet milling to obtain a particle size appropriate for tablet formation and for other formulation types. Finely divided (nanoparticulate) preparations of the active compounds in the present compositions can be prepared by processes known in the art, for example see International Patent Application Pub. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 100 to about 1000 mg of each of the active ingredients. For compositions containing emtricitabine, tenofovir disoproxil fumarate, efavirenz and a CCR5 antagonist, a unit dosage may contain, for example, from about 50 mg to about 500 mg, or from about 100 mg to about 400 mg of emtricitabine; from about 50 mg to about 500 mg, or from about 100 mg to about 400 mg of tenofovir disoproxil fumarate; from about 200 mg to about 1000 mg, or from about 400 mg to about 800 mg of efavirenz; and from about 10 mg to about 200 mg, or from about 10 mg to about 100 mg of a CCR5 antagonist.

For compositions containing lamivudine, zidovudine, efavirenz and a CCR5 antagonist, a unit dosage may contain, for example, from about 50 mg to about 500 mg, or from about 100 mg to about 400 mg of lamivudine; from about 100 mg to about 1000 mg, or from about 400 mg to about 800 mg of zidovudine; from about 200 mg to about 1000 mg, or from about 400 mg to about 800 mg of efavirenz; and from about 10 mg to about 200 mg, or from about 10 mg to about 100 mg of a CCR5 antagonist.

According to an embodiment, a unit dosage form containing emtricitabine, tenofovir disoproxil fumarate, efavirenz and a CCR5 antagonist comprises about 200 mg of emtricitabine, about 300 mg of tenofovir disoproxil fumarate about 600 mg of efavirenz, and from about 10 mg to about 100 mg of a CCR5 antagonist.

According to another embodiment, a unit dosage form containing lamivudine, zidovudine, efavirenz and a CCR5 antagonist comprises about 300 mg of lamivudine, about 600 mg of zidovudine, about 600 mg of efavirenz, and from about 10 mg to about 100 mg of a CCR5 antagonist.

The term “unit dosage forms” refers to physically discrete units suitable as unitary dosages for human subjects and other mammals, each unit containing a predetermined quantity of active materials calculated to produce the desired therapeutic effect, in association with a suitable pharmaceutical excipient.

The compositions according to the invention can be effective over a wide dosage range and are generally administered in a pharmaceutically effective amount. It will be understood, however, that the amount of the composition actually administered and the therapeutic regimen of administration will usually be determined by a physician, according to the relevant circumstances.

For preparing solid compositions such as tablets, the principal active ingredients may be mixed with a pharmaceutical excipient to form a solid preformulation composition containing a homogeneous mixture of the compounds in the composition. When referring to these preformulation compositions as homogeneous, the active ingredients are typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredients of the composition.

The tablets or pills containing a composition of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

The amount of the composition that is administered to a patient will vary depending upon what is being administered, the purpose of the administration, such as prophylaxis or therapy, the state of the patient, the manner of administration, and the like. In therapeutic applications, compositions can be administered to a patient already suffering from a HIV infection in an amount sufficient to cure or at least partially arrest the symptoms of the disease and its complications. In prophylactic applications, compositions can be administered to a patient who has been exposed or potentially exposed to HIV, wherein HIV infection is likely to occur absent effective intervention. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the composition preparations typically will be between 3 and 11, or from 5 to 9, or from 7 to 8. It will be understood that use of certain of the foregoing excipients, carriers, or stabilizers will result in the formation of pharmaceutical salts.

The therapeutic dosage of the compositions of the present invention can vary according to, for example, the particular use for which the treatment is made, the manner of administration of the composition, the health and condition of the patient, and the judgment of the prescribing physician. The proportion or concentration of the active compounds in the compositions of the invention can vary depending upon a number of factors including dosage, chemical characteristics (e.g., hydrophobicity), and the route of administration. For example, the compositions of the invention can be provided in an aqueous physiological buffer solution containing about 0.1 to about 10% w/v of the composition for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the CCR5 antagonist compound selected, formulation of the excipient, and its route of administration. Effective doses can be extrapolated from dose-response curves derived from in vitro or animal model test systems.

The compositions of the invention can also be formulated in combination with one or more additional active ingredients which can include any pharmaceutical agent such as anti-viral agents, antibodies, immune suppressants, anti-inflammatory agents and the like. In some embodiments, the compositions of the invention are formulated in combination with one or more anti-viral agents including protease inhibitors and other agents used for anti-HIV therapy.

Kits

The present invention also includes pharmaceutical kits useful, for example, in the treatment or prevention of HIV infection, which include one or more containers containing the pharmaceutical agents of the compositions of the invention, either together or separate and optionally in combination with at least one pharmaceutically acceptable carrier, and together comprising a therapeutically effective amount of a composition of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example A

### CCR5 Expression

A leukophoresis (Biological Specialty, Colmar, Pa.) was obtained from normal, drug free donors and peripheral blood mononuclear cells (PBMCs) were isolated via density gradient centrifugation. Monocytes were further isolated via centrifugal elutriation. After being washed, the monocytes were re-suspended at 10⁶ cells/ml with RPMI (Invitrogen, Carlsbad, Calif.) supplemented with 10% FBS (Hyclone, Logan, Utah) and 10-20 ng/mL of recombinant human IL-10 (R&D systems, Minneapolis, Minn.) and incubated in the same medium at 37° C. with 5% CO₂ for 24-48 hr. CCR5 expression on the IL-10-treated monocytes was then verified by staining the cells with a PE-conjugated anti-human CCR5 antibody ((PharMingen, San Diego, Calif.), followed by FACS analysis using FACSCalibur (BD Biosciences, Bedford, Mass.).

### Example B

### CCR5 Binding Assay

In a 96 well MULTISCREEN™ filter plate (Millipore Systems, Billerica, Mass.), 3×10⁵ IL-10-treated monocytes in 150 μL RPMI (Invitrogen, Carlsbad, Calif.) with 20 mM HEPES (Invitrogen, Carlsbad, Calif.) and 0.3% BSA (Sigma, St Louis, Mo.) were incubated at room temperature for 1 hr. with 0.2 nM ¹²⁵I-MIP-1β (Perkin Elmer, Boston, Mass.) and a series concentrations of compound of the invention. Non-specific binding was determined by incubating the cells with 0.3 μM MIP-1β (R&D Systems, Minneapolis, Minn.). The binding reaction was terminated by harvesting the cells onto the filter in the plate on a vacuum manifold (Millipore Systems, Billerica, Mass.). The filter was then washed 5 times with RPMI (Invitrogen, Carlsbad, Calif.) supplemented with 20 mM HEPES (Invitrogen, Carlsbad, Calif.), 0.3% BSA (Sigma, St Louis, Mo.) and 0.4 M NaCl on the vacuum manifold, air dried, and peeled from the plate. The filter dishes corresponding to the sample wells in a filter plate were punched out using the Millipore Punch System (Millipore Systems, Billerica, Mass.). The amount of bound radioactivity on each filter dish was determined by counting on a gamma counter. Specific binding was defined as the total binding minus the non-specific binding. The binding data were evaluated with Prism (GraphPad Software, San Diego, Calif.). Compounds of the invention were found to have a binding affinity of about 1 μM or less according to this assay.

### Example C

### HIV-1 Entry Assay

Replication defective HIV-1 reporter virions are generated by cotransfection of a plasmid encoding the NL4-3 strain of HIV-1 (which has been modified by mutation of the envelope gene and introduction of a luciferase reporter plasmid) along with a plasmid encoding one of several HIV-1 envelope genes as described by, for example, Connor et al, Virology, 206 (1995), 935-944. Following transfection of the two plasmids by calcium phosphate precipitation, the viral supernatants are harvested on day 3 and a functional viral titer determined. These stocks are then used to infect U87 cells stably expressing CD4 and the chemokine receptor CCR5 which have been preincubated with or without test compound. Infections are carried out for 2 hours at 37° C., the cells washed and media replaced with fresh media containing compound. The cells are incubated for 3 days, lysed and luciferase activity determined. Results are reported as the concentration of compound required to inhibit 50% of the luciferase activity in the control cultures.

### Example D

### HIV-1 Replication Assay in MT-4 Cells

Inhibition of HIV-1 NL4.3 (or III_{B}) replication assays can be carried out as previously described (Bridger, et al., J. Med. Chem. 42:3971-3981 (1999); De Clercq, et al., Proc. Natl. Acad. Sci. 89:5286-5290 (1992); De Clercq, et al., Antimicrob. Agents Chemother. 38:668-674 (1994); Bridger, et al. J. Med. Chem. 38:366-378 (1995)). To summarize, anti-HIV activity and cytotoxicity measurements are carried out in parallel and are based on the viability of MT-4 cells that are infected with HIV in the presence of various concentrations of the test compounds. After the MT-4 cells are allowed to proliferate for 5 days, the number of viable cells are quantified by a tetrazolium-based calorimetric 3-(4,5-dimethylthiazol-2-yl)-2,5-diphenyltetrazolium bromide (MTT) procedure in 96-well microtrays. Results can be quantified to yield EC₅₀ values which represent the concentration required to protect 50% of the virus-infected cells against viral cytopathicity.

### Example E

### Chemokine Receptor Inhibition/Binding Assays

The capacity of the compounds of the invention to antagonize chemokine receptor (e.g., CCR2) function can be determined using a suitable screen (e.g., high through-put assay). For example, an agent can be tested in an extracellular acidification assay, calcium flux assay, ligand binding assay or chemotaxis assay (see, for example, Hesselgesser et al., J Biol. Chem. 273(25):15687-15692 (1998); WO 00/05265 and WO 98/02151, each of which is incorporated herein by reference in its entirety).

In an example assay, a chemokine receptor which can be isolated or recombinantly derived is used which has at least one property, activity or functional characteristic of a mammalian chemokine receptor. The specific property can be a binding property (to, for example, a ligand or inhibitor), a signaling activity (e.g., activation of a mammalian G protein, induction of rapid and transient increase in the concentration of cytosolic free calcium [Ca⁺⁺]i, cellular response function (e.g., stimulation of chemotaxis or inflammatory mediator release by leukocytes), and the like.

In one embodiment, a composition containing a chemokine receptor or variant thereof is maintained under conditions suitable for binding. The receptor is contacted with a compound to be tested, and binding is detected or measured.

In further embodiments, the assay is a cell-based assay in which cells are used that are stably or transiently transfected with a vector or expression cassette having a nucleic acid sequence which encodes the receptor. The cells are maintained under conditions appropriate for expression of the receptor and are contacted with an agent under conditions appropriate for binding to occur. Binding can be detected using standard techniques. For example, the extent of binding can be determined relative to a suitable control. Also, a cellular fraction, such as a membrane fraction, containing the receptor can be used in lieu of whole cells.

Detection of binding or complex formation between compounds of the invention and chemokine receptors can be detected directly or indirectly. For example, the compound can be labeled with a suitable label (e.g., fluorescent label, label, isotope label, enzyme label, and the like) and binding can be determined by detection of the label. Specific and/or competitive binding can be assessed by competition or displacement studies, using unlabeled agent or a ligand as a competitor.

The antagonist activity of test agents can be reported as the inhibitor concentration required for 50% inhibition (IC₅₀ values) of specific binding in receptor binding assays using, for example, ¹²⁵I-labeled MCP-1, as ligand, and Peripheral Blood Mononuclear Cells (PBMCs) prepared from normal human whole blood via density gradient centrifugation. Specific binding is preferably defined as the total binding (e.g., total cpm on filters) minus the non-specific binding. Non-specific binding is defined as the amount of cpm still detected in the presence of excess unlabeled competitor (e.g., MCP-1).

The human PBMCs described above can be used in a suitable binding assay. For example, 200,000 to 500,000 cells can be incubated with 0.1 to 0.2 nM ¹²⁵I-labeled MCP-1, with or without unlabeled competitor (10 nM MCP-1) or various concentrations of compounds to be tested. ¹²⁵I-labeled MCP-1, can be prepared by suitable methods or purchased from commercial vendors (Perkin Elmer, Boston Mass.), The binding reactions can be performed in 50 to 250 μl of a binding buffer consisting of 1M HEPES pH 7.2, and 0.1% BSA (bovine serum albumin), for 30 min at room temperature. The binding reactions can be terminated by harvesting the membranes by rapid filtration through glass fiber filters (Perkin Elmer) which can be presoaked in 0.3% polyethyleneimine or Phosphate Buffered Saline (PBS). The filters can be rinsed with approximately 600 μL of binding buffer containing 0.5 M NaCl or PBS, then dried, and the amount of bound radioactivity can be determined by counting on a Gamma Counter (Perkin Elmer).

The capacity of compounds to antagonize chemokine receptor function can also be determined in a leukocyte chemotaxis assay using suitable cells. Suitable cells include, for example, cell lines, recombinant cells or isolated cells which express a chemokine receptor (e.g., CCR2) and undergo chemokine receptor ligand-induced (e.g., MCP-1) chemotaxis. The assay utilizes human peripheral blood mononuclear cells, in a modified Boyden Chamber (Neuro Probe). 500,000 cells in serum free DMEM media (In Vitrogen) are incubated with or without the inhibitors and warmed to 37° C. The chemotaxis chamber (Neuro Probe) is also prewarmed. Warmed 10 nM MCP-1 (400 μL) is added to the bottom chamber in all wells expect the negative control which has DMEM added. An 8 micron membrane filter (Neuro Probe) is place on top and the chamber lid is closed. Cells are then added to the holes in the chamber lid which are associated with the chamber wells below the filter membrane. The whole chamber is incubated at 37° C., 5% CO₂ for 30 minutes. The cells are then aspirated off, the chamber lid opened, and the filter gently removed. The top of the filter is washed 3 times with PBS and the bottom is left untouched. The filter is air dried and stained with Wright Geimsa stain (Sigma). Filters are counted by microscopy. The negative control wells serve as background and are subtracted from all values. Antagonist potency can be determined by comparing the number of cells that migrate to the bottom chamber in wells which contain antagonist, to the number of cells which migrate to the bottom chamber in MCP-1 control wells.

Compounds of the present invention can be considered active if they have IC₅₀ values in the range of about 0.01 to about 500 nM for the above binding assay. In chemotaxis assays, active compounds have IC₅₀ values in the range of about 1 to about 3000 nM.

### Example F

### Assay to Test the Efficacy/Synergy of Combinations of Antiretroviral Medicines Containing CCR5 Antagonists

Combinations of antiviral agents can be examined for their combined ability to inhibit HIV replication in cell based assay systems. Typically, two (or more) compounds or two (or more) mixtures of compounds (e.g., fixed dose mixtures) are combined at concentrations near their experimentally determined 50% inhibitory concentrations (IC₅₀). The mixtures are serially diluted 32-fold using two-fold dilutions. This dilution set would be designated the 1:1 dilution. Additional dilution series are prepared where the relative concentrations are 10:1, 3:1, 1:3 and 1:10. The dilution series are then tested for their ability to inhibit virus replication. For combinations containing a CCR5 inhibitor, a CCR5-tropic virus and a CCR5 receptor-bearing cell line are used; typically the Bal-1 virus and peripheral blood mononuclear cells (PBMC) are used. A parameter designated the “combination index” (CI) is calculated from the virus inhibition data using the Chou and Talalay model for drug combinations (Chou, T C and Talalay P (1984) Quantitative analysis of dose-effect relationships, the combined effect of multiple drugs or enzyme inhibitors. Advances in Enzyme Regulation 22:27-57). According to the Chou-Talalay model, CI values less than 0.8 are designated “synergistic,” CI values between 0.8 and 1.2 are designated “additive,” and CI values >1.2 are designated antagonistic. Commercial computer programs such as the CalcuSyn Software (Biosoft) can be used to determine the CI values.

The pairwise combination assay method with CI measurement can also be carried out using binding to enzyme or receptor instead of inhibition of replication. For CCR5 antagonists, pairwise combinations of different CCR5 antagonists could be examined for their ability to inhibit ligand binding to CCR5 receptor.

Examples of true antagonism between antiretroviral agents used in the treatment of HIV include the combination of the two thymidine nucleoside analogs d4T and AZT. Because AZT prevents the phosphorylation of d4T, and also exhibits feed-forward inhibition of its own phosphorylation, this combination is antagonistic in vitro, with typical CI values of 2 to 3 being observed.

A number of embodiments of the invention have been described. Nevertheless, it will be understood that various modifications may be made without departing from the spirit and scope of the invention. Each reference, including all patent, patent applications, and publications, cited in the present application is incorporated herein by reference in its entirety. Accordingly, other embodiments are within the scope of the following claims.

What is claimed is:

1. A pharmaceutical composition comprising:
(A) emtricitabine;
(B) tenofovir disoproxil fumarate;
(C) efavirenz; and
(D) a CCR5 antagonist.

2. The pharmaceutical composition according to claim 1, comprising a mixture of amounts of (A), (B), (C) and (D) that is therapeutically effective for treating an HIV infection in a person.

3. The pharmaceutical composition according to claim 1 in combination with at least one pharmaceutically acceptable carrier.

4. The pharmaceutical composition according to claim 1, wherein the CCR5 antagonist comprises a compound of Formula I: 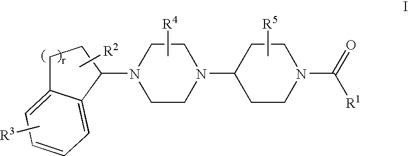
or pharmaceutically acceptable salt thereof, wherein:
R¹ is heteroaryl optionally substituted by one or more R⁶;
R² is H, halo, cyano, nitro, C₁-C₆ alkyl, C₁-C₆ haloalkyl, C₂-C₆ alkenyl, C₂-C₆ alkynyl, aryl, heteroaryl, C₃-C₇ cycloalkyl, heterocycloalkyl, SOR⁷, SO₂R⁷, COR⁸, OR⁹, SR⁹, COOR⁹, NR¹⁰R¹¹ or NR¹⁰COR⁸;
R³ is F, Cl, Br, I, C₁-C₄ haloalkyl, C₁-C₄ haloalkoxy or heteroaryl;
R⁴ is H, C₁-C₆ alkyl, C₂-C₆ alkenyl, C₂-C₆ alkynyl or C₁-C₆ haloalkyl;
R⁵ is H, C₁-C₆ alkyl, C₂-C₆ alkenyl, C₂-C₆ alkynyl or C₁-C₆ haloalkyl;
R⁶ is H, C₁-C₆ alkyl, C₂-C₆ alkenyl, C₂-C₆ alkynyl, C₁-C₆ haloalkyl, C₁-C₆ alkoxy, C₁-C₆ haloalkoxy, amino, (C₁-C₆ alkyl)amino or di(C₁-C₆ alkyl)amino;
R⁷ is H, C₁-C₆ alkyl, C₂-C₆ alkenyl, C₂-C₆ alkynyl, C₁-C₆ haloalkyl, aryl, heteroaryl, C₃-C₇ cycloalkyl, heterocycloalkyl, arylalkyl, heteroarylalkyl, (C₃-C₇ cycloalkyl)alkyl, heterocycloalkylalkyl, or NR¹²R¹³;
R⁸ is H, C₁-C₆ alkyl, C₂-C₆ alkenyl, C₂-C₆ alkynyl, C₁-C₆ haloalkyl, aryl, heteroaryl, C₃-C₇ cycloalkyl, heterocycloalkyl, arylalkyl, heteroarylalkyl, (C₃-C₇ cycloalkyl)alkyl, heterocycloalkylalkyl, or NR¹²R¹³;
R⁹ is H, C₁-C₆ alkyl, C₂-C₆ alkenyl, C₂-C₆ alkynyl, C₁-C₆ haloalkyl, alkoxyalkyl, haloalkoxyalkyl, aryloxyalkyl, heteroaryloxyalkyl, cycloalkyloxyalkyl, heterocycloalkyloxyalkyl, aryl, heteroaryl, C₃-C₇ cycloalkyl, heterocycloalkyl, arylalkyl, heteroarylalkyl; (C₃-C₇ cycloalkyl)alkyl or heterocycloalkylalkyl;
R¹⁰ and R¹¹ are each, independently, H, C₁-C₆ alkyl, C₂-C₆ alkenyl, C₂-C₆ alkynyl, C₁-C₆ haloalkyl, aryl, heteroaryl, C₃-C₇ cycloalkyl, heterocycloalkyl, arylalkyl, heteroarylalkyl; (C₃-C₇ cycloalkyl)alkyl or heterocycloalkylalkyl;
or R¹⁰ and R¹¹ together with the N atom to which they are attached form a 3-, 4-, 5-, 6-, or 7-membered heterocycloalkyl group;
R¹² and R¹³ are each, independently, H, C₁-C₆ alkyl, C₂-C₆ alkenyl, C₂-C₆ alkynyl, C₁-C₆ haloalkyl, aryl, heteroaryl, C₃-C₇ cycloalkyl, heterocycloalkyl, arylalkyl, heteroarylalkyl; (C₃-C₇ cycloalkyl)alkyl or heterocycloalkylalkyl;
or R¹² and R¹³ together with the N atom to which they are attached form a 3-, 4-, 5-, 6-, or 7-membered heterocycloalkyl group;
r is 1, 2 or 3.

5. The pharmaceutical composition according to claim 4 wherein R¹ is a 5-, 6-, 9- or 10-membered heteroaryl group containing at least one ring-forming N atom, wherein said 5-, 6-, 9- or 10-membered heteroaryl group is optionally substituted by 1, 2, 3 or 4 R⁶ groups.

6. The pharmaceutical composition according to claim 4 wherein R² is H, C₁-C₆ alkyl, C₁-C₆ haloalkyl, OR⁹, SR⁹ or NR¹⁰R¹¹.

7. The pharmaceutical composition according to claim 4 wherein R³ is F, Br, CF₃, or 6- or 5-membered heteroaryl.

8. The pharmaceutical composition according to claim 4 wherein R⁴ is C₁-C₆ alkyl.

9. The pharmaceutical composition according to claim 4 wherein R⁵ is C₁-C₆ alkyl.

10. The pharmaceutical composition according to claim 4, wherein the compound of Formula I is a compound having Formula IIa: 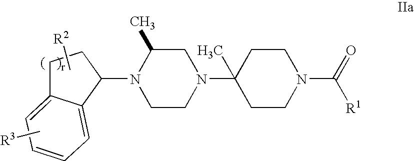
or pharmaceutically acceptable salt form thereof.

11. The pharmaceutical composition according to claim 4, wherein the compound according to Formula I is selected from:
5-({4-[(3S)-4-(5-bromo-2,3-dihydro-1H-inden-1-yl)-3-methylpiperazin-1-yl]-4-methylpiperidin-1-yl}carbonyl)-4,6-dimethylpyrimidine;
5-({4-[(3S)-4-(5-fluoro-2,3-dihydro-1H-inden-1-yl)-3-methylpiperazin-1-yl]-4-methylpiperidin-1-yl}carbonyl)-4,6-dimethylpyrimidine;
5-({4-[(3S)-4-(6-bromo-2,3-dihydro-1H-inden-1-yl)-3-methylpiperazin-1-yl]-4-methylpiperidin-1-yl}carbonyl)-4,6-dimethylpyrimidine;
5-({4-[(3S)-4-(6-fluoro-2,3-dihydro-1H-inden-1-yl)-3-methylpiperazin-1-yl]-4-methylpiperidin-1-yl}carbonyl)-4,6-dimethylpyrimidine;
5-({4-[(3S)-4-(6-bromo-1,2,3,4-tetrahydronaphthalen-1-yl)-3-methylpiperazin-1-yl]-4-methylpiperidin-1-yl}carbonyl)-4,6-dimethylpyrimidine;
5-({4-[(3S)-4-(7-bromo-1,2,3,4-tetrahydronaphthalen-1-yl)-3-methylpiperazin-1-yl]-4-methylpiperidin-1-yl}carbonyl)-4,6-dimethylpyrimidine;
4,6-dimethyl-5-[(4-methyl-4-{(3S)-3-methyl-4-[6-(trifluoromethyl)-2,3-dihydro-1H-inden-1-yl]piperazin-1-yl}piperidin-1-yl)carbonyl]pyrimidine;
4,6-dimethyl-5-[(4-methyl-4-{(3S)-3-methyl-4-[5-(trifluoromethyl)-2,3-dihydro-1H-inden-1-yl]piperazin-1-yl}piperidin-1-yl)carbonyl]pyrimidine;
1-((2S)-4-{1-[(4,6-dimethylpyrimidin-5-yl)carbonyl]-4-methylpiperidin-4-yl}-2-methylpiperazin-1-yl)-5-(trifluoromethyl)indan-2-ol;
5-[(4-{(3S)-4-[2-methoxy-5-(trifluoromethyl)-2,3-dihydro-1H-inden-1-yl]-3-methylpiperazin-1-yl}-4-methylpiperidin-1-yl)carbonyl]-4,6-dimethylpyrimidine;
5-[(4-(3S)-4-[(1R,2R)-2-ethoxy-5-(trifluoromethyl)-2,3-dihydro-1H-inden-1-yl]-3-methylpiperazin-1-yl-4-methylpiperidin-1-yl)carbonyl]-4,6-dimethylpyrimidine;
5-[(4-{(3S)-4-[(1R,2R)-2-(2-methoxyethoxy)-5-(trifluoromethyl)-2,3-dihydro-1H-inden-1-yl]-3-methylpiperazin-1-yl}-4-methylpiperidin-1-yl)carbonyl]-4,6-dimethylpyrimidine;
4-[(4-{(3S)-4-[(1S,2R)-2-ethoxy-5-(trifluoromethyl)-2,3-dihydro-1H-inden-1-yl]-3-methylpiperazin-1-yl}-4-methylpiperidin-1-yl)carbonyl]cinnoline;
4-[(4-{(3S)-4-[(1R,2R)-2-ethoxy-5-(trifluoromethyl)-2,3-dihydro-1H-inden-1-yl]-3-methylpiperazin-1-yl}-4-methylpiperidin-1-yl)carbonyl]quinoline;
5-[(4-{(3S)-4-[(1R,2R)-2-ethoxy-5-(trifluoromethyl)-2,3-dihydro-1H-inden-1-yl]-3-methylpiperazin-1-yl}-4-methylpiperidin-1-yl)carbonyl]quinoline;
4-[(4-{(3S)-4-[(1R,2R)-2-ethoxy-5-(trifluoromethyl)-2,3-dihydro-1H-inden-1-yl]-3-methylpiperazin-1-yl}-4-methylpiperidin-1-yl)carbonyl]-1,8-naphthyridine;
5-[(4-{(3S)-4-[(1R,2R)-2-ethoxy-5-(trifluoromethyl)-2,3-dihydro-1H-inden-1-yl]-3-methylpiperazin-1-yl}-4-methylpiperidin-1-yl)carbonyl]isoquinoline;
5-[(4-{(3S)-4-[(1R,2R)-5-bromo-2-ethoxy-2,3-dihydro-1H-inden-1-yl]-3-methylpiperazin-1-yl}-4-methylpiperidin-1-yl)carbonyl]-4,6-dimethylpyrimidine;
4-[(4-{(3S)-4-[(1R,2R)-5-bromo-2-ethoxy-2,3-dihydro-1H-inden-1-yl]-3-methylpiperazin-1-yl}-4-methylpiperidin-1-yl)carbonyl]cinnoline;
4-[(4-{(3S)-4-[(1R,2R)-5-bromo-2-ethoxy-2,3-dihydro-1H-inden-1-yl]-3-methylpiperazin-1-yl}-4-methylpiperidin-1-yl)carbonyl]-1,8-naphthyridine;
5-[(4-{(3S)-4-[(1R,2R)-5-bromo-2-(pyridin-2-yloxy)-2,3-dihydro-1H-inden-1-yl]-3-methylpiperazin-1-yl}-4-methylpiperidin-1-yl)carbonyl]-4,6-dimethylpyrimidine;
5-[(4-{(3S)-4-[(1R,2R)-2-ethoxy-5-(1,3-thiazol-2-yl)-2,3-dihydro-1H-inden-1-yl]-3-methylpiperazin-1-yl}-4-methylpiperidin-1-yl)carbonyl]-4,6-dimethylpyrimidine;
5-[(4-{(3S)-4-[(1R,2R)-2-ethoxy-5-pyridin-2-yl-2,3-dihydro-1H-inden-1-yl]-3-methylpiperazin-1-yl}-4-methylpiperidin-1-yl)carbonyl]-4,6-dimethylpyrimidine; and
5-[(4-{(3S)-4-[3-methoxy-5-(trifluoromethyl)-2,3-dihydro-1H-inden-1-yl]-3-methylpiperazin-1-yl}-4-methylpiperidin-1-yl)carbonyl]-4,6-dimethylpyrimidine;
and pharmaceutically acceptable salts thereof.

12. The pharmaceutical composition according to claim 4, wherein the compound according to Formula I is selected from:
5-[(4-{(3S)-4-[2-methoxy-5-(trifluoromethyl)-2,3-dihydro-1H-inden-1-yl]-3-methylpiperazin-1-yl}-4-methylpiperidin-1-yl)carbonyl]-4,6-dimethylpyrimidine;
5-[(4-(3S)-4-[(1R,2R)-2-ethoxy-5-(trifluoromethyl)-2,3-dihydro-1H-inden-1-yl]-3-methylpiperazin-1-yl-4-methylpiperidin-1-yl)carbonyl]-4,6-dimethylpyrimidine; and
5-[(4-{(3S)-4-[(1R,2R)-2-ethoxy-5-(1,3-thiazol-2-yl)-2,3-dihydro-1H-inden-1-yl]-3-methylpiperazin-1-yl}-4-methylpiperidin-1-yl)carbonyl]-4,6-dimethylpyrimidine; and
and pharmaceutically acceptable salts thereof.

13. The pharmaceutical composition according to claim 1, wherein said composition comprises a solid dosage form.

14. The pharmaceutical composition according to claim 13, wherein said solid dosage form is suitable for oral administration.

15. A pharmaceutical composition consisting essentially of:
(A) emtricitabine;
(B) tenofovir disoproxil fumarate;
(C) efavirenz;
(D) a CCR5 antagonist; and
(E) one or more pharmaceutically acceptable carriers.

16. A method for treating an HIV infection in a person, comprising administering to the person a therapeutically effective amount of a pharmaceutical composition according to claim 1.

17. The method of claim 16, wherein the person who is treated has not previously received antiretroviral therapy.

18. The method according to claim 16, wherein the pharmaceutical composition comprises a solid dosage form suitable for oral administration.

19. The method of claim 16 wherein the pharmaceutical composition is administered to said person once per day.

20. A method for treating an HIV infection in a person who has not previously received antiretroviral therapy to treat said infection, comprising administering to said person, separately or together, therapeutically effective amounts of the pharmaceutical agents:
(A) emtricitabine;
(B) tenofovir disoproxil fumarate;
(C) efavirenz; and
(D) at least one CCR5 antagonist selected from:
i) 5-[(4-{(3S)-4-[2-methoxy-5-(trifluoromethyl)-2,3-dihydro-1H-inden-1-yl]-3-methylpiperazin-1-yl}-4-methylpiperidin-1-yl)carbonyl]-4,6-dimethylpyrimidine;
ii) 5-[(4-(3S)-4-[(1R,2R)-2-ethoxy-5-(trifluoromethyl)-2,3-dihydro-1H-inden-1-yl]-3-methylpiperazin-1-yl-4-methylpiperidin-1-yl)carbonyl]-4,6-dimethylpyrimidine; and
iii) 5-[(4-{(3S)-4-[(1R,2R)-2-ethoxy-5-(1,3-thiazol-2-yl)-2,3-dihydro-1H-inden-1-yl]-3-methylpiperazin-1-yl}-4-methylpiperidin-1-yl)carbonyl]-4,6-dimethylpyrimidine;
or pharmaceutically acceptable salts thereof.

21. The method of claim 20 wherein said pharmaceutical agents of (A), (B), (C), and (D) are administered together in a pharmaceutical composition provided in a solid dosage form suitable for oral administration.

22. The method of claim 20 wherein said pharmaceutical agents (A), (B), (C), and (D) are administered to said patient once per day.

23. A method for treating an HIV infection in a person, comprising administering to the person, separately or together, therapeutically effective amounts of:
(A) emtricitabine;
(B) tenofovir disoproxil fumarate;
(C) efavirenz; and
(D) a CCR5 antagonist.

24. A pharmaceutical composition comprising:
(A) lamivudine;
(B) zidovudine;
(C) efavirenz; and
(D) a CCR5 antagonist.

25. The pharmaceutical composition according to claim 24, comprising a mixture of amounts of (A), (B), (C) and (D) that is therapeutically effective for treating an HIV infection in a person.

26. The pharmaceutical composition according to claim 24 in combination with at least one pharmaceutically acceptable carrier.

27. The pharmaceutical composition according to claim 24, wherein the CCR5 antagonist comprises a compound of Formula I: 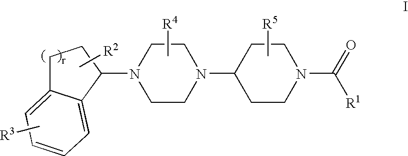
or pharmaceutically acceptable salt thereof, wherein:
R¹ is heteroaryl optionally substituted by one or more R⁶;
R² is H, halo, cyano, nitro, C₁-C₆ alkyl, C₁-C₆ haloalkyl, C₂-C₆ alkenyl, C₂-C₆ alkynyl, aryl, heteroaryl, C₃-C₇ cycloalkyl, heterocycloalkyl, SOR⁷, SO₂R⁷, COR⁸, OR⁹, SR⁹, COOR⁹, NR¹⁰R¹¹ or NR¹⁰COR⁸;
R³ is F, Cl, Br, I, C₁-C₄ haloalkyl, C₁-C₄ haloalkoxy or heteroaryl;
R⁴ is H, C₁-C₆ alkyl, C₂-C₆ alkenyl, C₂-C₆ alkynyl or C₁-C₆ haloalkyl;
R⁵ is H, C₁-C₆ alkyl, C₂-C₆ alkenyl, C₂-C₆ alkynyl or C₁-C₆ haloalkyl;
R⁶ is H, C₁-C₆ alkyl, C₂-C₆ alkenyl, C₂-C₆ alkynyl, C₁-C₆ haloalkyl, C₁-C₆ alkoxy, C₁-C₆ haloalkoxy, amino, (C₁-C₆ alkyl)amino or di(C₁-C₆ alkyl)amino;
R⁷ is H, C₁-C₆ alkyl, C₂-C₆ alkenyl, C₂-C₆ alkynyl, C₁-C₆ haloalkyl, aryl, heteroaryl, C₃-C₇ cycloalkyl, heterocycloalkyl, arylalkyl, heteroarylalkyl, (C₃-C₇ cycloalkyl)alkyl, heterocycloalkylalkyl, or NR¹²R¹³;
R⁸ is H, C₁-C₆ alkyl, C₂-C₆ alkenyl, C₂-C₆ alkynyl, C₁-C₆ haloalkyl, aryl, heteroaryl, C₃-C₇ cycloalkyl, heterocycloalkyl, arylalkyl, heteroarylalkyl, (C₃-C₇ cycloalkyl)alkyl, heterocycloalkylalkyl, or NR¹²R¹³;
R⁹ is H, C₁-C₆ alkyl, C₂-C₆ alkenyl, C₂-C₆ alkynyl, C₁-C₆ haloalkyl, alkoxyalkyl, haloalkoxyalkyl, aryloxyalkyl, heteroaryloxyalkyl, cycloalkyloxyalkyl, heterocycloalkyloxyalkyl, aryl, heteroaryl, C₃-C₇ cycloalkyl, heterocycloalkyl, arylalkyl, heteroarylalkyl; (C₃-C₇ cycloalkyl)alkyl or heterocycloalkylalkyl;
R¹⁰ and R¹¹ are each, independently, H, C₁-C₆ alkyl, C₂-C₆ alkenyl, C₂-C₆ alkynyl, C₁-C₆ haloalkyl, aryl, heteroaryl, C₃-C₇ cycloalkyl, heterocycloalkyl, arylalkyl, heteroarylalkyl; (C₃-C₇ cycloalkyl)alkyl or heterocycloalkylalkyl;
or R¹⁰ and R¹¹ together with the N atom to which they are attached form a 3-, 4-, 5-, 6-, or 7-membered heterocycloalkyl group;
R¹² and R¹³ are each, independently, H, C₁-C₆ alkyl, C₂-C₆ alkenyl, C₂-C₆ alkynyl, C₁-C₆ haloalkyl, aryl, heteroaryl, C₃-C₇ cycloalkyl, heterocycloalkyl, arylalkyl, heteroarylalkyl; (C₃-C₇ cycloalkyl)alkyl or heterocycloalkylalkyl;
or R¹² and R¹³ together with the N atom to which they are attached form a 3-, 4-, 5-, 6-, or 7-membered heterocycloalkyl group;
r is 1, 2 or 3.

28. The pharmaceutical composition according to claim 27 wherein R¹ is a 5-, 6-, 9- or 10-membered heteroaryl group containing at least one ring-forming N atom, wherein said 5-, 6-, 9- or 10-membered heteroaryl group is optionally substituted by 1, 2, 3 or 4 R⁶ groups.

29. The pharmaceutical composition according to claim 27 wherein R² is H, C₁-C₆ alkyl, C₁-C₆ haloalkyl, OR⁹, SR⁹ or NR¹⁰R¹¹.

30. The pharmaceutical composition according to claim 27 wherein R³ is F, Br, CF₃, or 6- or 5-membered heteroaryl.

31. The pharmaceutical composition according to claim 27 wherein R⁴ is C₁-C₆ alkyl.

32. The pharmaceutical composition according to claim 27 wherein R⁵ is C₁-C₆ alkyl.

33. The pharmaceutical composition according to claim 27, wherein the compound of Formula I is a compound having Formula IIa: 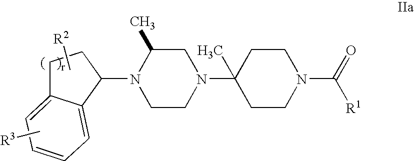
or pharmaceutically acceptable salt form thereof.

34. The pharmaceutical composition according to claim 27, wherein the compound according to Formula I is selected from:
5-({4-[(3S)-4-(5-bromo-2,3-dihydro-1H-inden-1-yl)-3-methylpiperazin-1-yl]-4-methylpiperidin-1-yl}carbonyl)-4,6-dimethylpyrimidine;
5-({4-[(3S)-4-(5-fluoro-2,3-dihydro-1H-inden-1-yl)-3-methylpiperazin-1-yl]-4-methylpiperidin-1-yl}carbonyl)-4,6-dimethylpyrimidine;
5-({4-[(3S)-4-(6-bromo-2,3-dihydro-1H-inden-1-yl)-3-methylpiperazin-1-yl]-4-methylpiperidin-1-yl}carbonyl)-4,6-dimethylpyrimidine;
5-({4-[(3S)-4-(6-fluoro-2,3-dihydro-1H-inden-1-yl)-3-methylpiperazin-1-yl]-4-methylpiperidin-1-yl}carbonyl)-4,6-dimethylpyrimidine;
5-({4-[(3S)-4-(6-bromo-1,2,3,4-tetrahydronaphthalen-1-yl)-3-methylpiperazin-1-yl]-4-methylpiperidin-1-yl}carbonyl)-4,6-dimethylpyrimidine;
5-({4-[(3S)-4-(7-bromo-1,2,3,4-tetrahydronaphthalen-1-yl)-3-methylpiperazin-1-yl]-4-methylpiperidin-1-yl}carbonyl)-4,6-dimethylpyrimidine;
4,6-dimethyl-5-[(4-methyl-4-{(3S)-3-methyl-4-[6-(trifluoromethyl)-2,3-dihydro-1H-inden-1-yl]piperazin-1-yl}piperidin-1-yl)carbonyl]pyrimidine;
4,6-dimethyl-5-[(4-methyl-4-{(3S)-3-methyl-4-[5-(trifluoromethyl)-2,3-dihydro-1H-inden-1-yl]piperazin-1-yl}piperidin-1-yl)carbonyl]pyrimidine;
1-((2S)-4-{1-[(4,6-dimethylpyrimidin-5-yl)carbonyl]-4-methylpiperidin-4-yl}-2-methylpiperazin-1-yl)-5-(trifluoromethyl)indan-2-ol;
5-[(4-{(3S)-4-[2-methoxy-5-(trifluoromethyl)-2,3-dihydro-1H-inden-1-yl]-3-methylpiperazin-1-yl}-4-methylpiperidin-1-yl)carbonyl]-4,6-dimethylpyrimidine;
5-[(4-(3S)-4-[(1R,2R)-2-ethoxy-5-(trifluoromethyl)-2,3-dihydro-1H-inden-1-yl]-3-methylpiperazin-1-yl-4-methylpiperidin-1-yl)carbonyl]-4,6-dimethylpyrimidine;
5-[(4-{(3S)-4-[(1R,2R)-2-(2-methoxyethoxy)-5-(trifluoromethyl)-2,3-dihydro-1H-inden-1-yl]-3-methylpiperazin-1-yl}-4-methylpiperidin-1-yl)carbonyl]-4,6-dimethylpyrimidine;
4-[(4-{(3S)-4-[(1S,2R)-2-ethoxy-5-(trifluoromethyl)-2,3-dihydro-1H-inden-1-yl]-3-methylpiperazin-1-yl}-4-methylpiperidin-1-yl)carbonyl]cinnoline;
4-[(4-{(3S)-4-[(1R,2R)-2-ethoxy-5-(trifluoromethyl)-2,3-dihydro-1H-inden-1-yl]-3-methylpiperazin-1-yl}-4-methylpiperidin-1-yl)carbonyl]quinoline;
5-[(4-{(3S)-4-[(1R,2R)-2-ethoxy-5-(trifluoromethyl)-2,3-dihydro-1H-inden-1-yl]-3-methylpiperazin-1-yl}-4-methylpiperidin-1-yl)carbonyl]quinoline;
4-[(4-{(3S)-4-[(1R,2R)-2-ethoxy-5-(trifluoromethyl)-2,3-dihydro-1H-inden-1-yl]-3-methylpiperazin-1-yl}-4-methylpiperidin-1-yl)carbonyl]-1,8-naphthyridine;
5-[(4-{(3S)-4-[(1,R2R)-2-ethoxy-5-(trifluoromethyl)-2,3-dihydro-1H-inden-1-yl]-3-methylpiperazin-1-yl}-4-methylpiperidin-1-yl)carbonyl]isoquinoline;
5-[(4-{(3S)-4-[(1R,2R)-5-bromo-2-ethoxy-2,3-dihydro-1H-inden-1-yl]-3-methylpiperazin-1-yl}-4-methylpiperidin-1-yl)carbonyl]-4,6-dimethylpyrimidine;
4-[(4-{(3S)-4-[(1R,2R)-5-bromo-2-ethoxy-2,3-dihydro-1H-inden-1-yl]-3-methylpiperazin-1-yl}-4-methylpiperidin-1-yl)carbonyl]cinnoline;
4-[(4-{(3S)-4-[(1R,2R)-5-bromo-2-ethoxy-2,3-dihydro-1H-inden-1-yl]-3-methylpiperazin-1-yl}-4-methylpiperidin-1-yl)carbonyl]-1,8-naphthyridine;
5-[(4-{(3S)-4-[(1R,2R)-5-bromo-2-(pyridin-2-yloxy)-2,3-dihydro-1H-inden-1-yl]-3-methylpiperazin-1-yl}-4-methylpiperidin-1-yl)carbonyl]-4,6-dimethylpyrimidine;
5-[(4-{(3S)-4-[(1R,2R)-2-ethoxy-5-(1,3-thiazol-2-yl)-2,3-dihydro-1H-inden-1-yl]-3-methylpiperazin-1-yl}-4-methylpiperidin-1-yl)carbonyl]-4,6-dimethylpyrimidine;
5-[(4-{(3S)-4-[(1R,2R)-2-ethoxy-5-pyridin-2-yl-2,3-dihydro-1H-inden-1-yl]-3-methylpiperazin-1-yl}-4-methylpiperidin-1-yl)carbonyl]-4,6-dimethylpyrimidine; and
5-[(4-{(3S)-4-[3-Methoxy-5-(trifluoromethyl)-2,3-dihydro-1H-inden-1-yl]-3-methylpiperazin-1-yl}-4-methylpiperidin-1-yl)carbonyl]-4,6-dimethylpyrimidine; and
pharmaceutically acceptable salts thereof.

35. The pharmaceutical composition according to claim 27, wherein the compound according to Formula I is selected from:
5-[(4-{(3S)-4-[2-methoxy-5-(trifluoromethyl)-2,3-dihydro-1H-inden-1-yl]-3-methylpiperazin-1-yl}-4-methylpiperidin-1-yl)carbonyl]-4,6-dimethylpyrimidine;
5-[(4-(3S)-4-[(1R,2R)-2-ethoxy-5-(trifluoromethyl)-2,3-dihydro-1H-inden-1-yl]-3-methylpiperazin-1-yl-4-methylpiperidin-1-yl)carbonyl]-4,6-dimethylpyrimidine; and
5-[(4-{(3S)-4-[(1R,2R)-2-ethoxy-5-(1,3-thiazol-2-yl)-2,3-dihydro-1H-inden-1-yl]-3-methylpiperazin-1-yl}-4-methylpiperidin-1-yl)carbonyl]-4,6-dimethylpyrimidine; and
pharmaceutically acceptable salts thereof.

36. The pharmaceutical composition according to claim 27, wherein said composition comprises a solid dosage form suitable for oral administration.

37. A pharmaceutical composition consisting essentially of:
(A) lamivudine;
(B) zidovudine;
(C) efavirenz;
(D) a CCR5 antagonist; and
(E) one or more pharmaceutically acceptable carriers.

38. A method for treating an HIV infection in a person, comprising administering to the person a therapeutically effective amount of a pharmaceutical composition according to claim 24.

39. The method of claim 38, wherein the person who is treated has not previously received antiretroviral therapy.

40. The method according to claim 38, wherein the pharmaceutical composition comprises a solid dosage form suitable for oral administration.

41. The method of claim 38 wherein the pharmaceutical composition is administered to said person once per day.

42. A method for treating an HIV infection in a person who has not previously received antiretroviral therapy to treat said infection, comprising administering to said person, separately or together, therapeutically effective amounts of the pharmaceutical agents:
(A) lamivudine;
(B) zidovudine;
(C) efavirenz; and
(D) at least one CCR5 antagonist selected from:
i) 5-[(4-{(3S)-4-[2-methoxy-5-(trifluoromethyl)-2,3-dihydro-1H-inden-1-yl]-3-methylpiperazin-1-yl}-4-methylpiperidin-1-yl)carbonyl]-4,6-dimethylpyrimidine;
ii) 5-[(4-(3S)-4-[(1R,2R)-2-ethoxy-5-(trifluoromethyl)-2,3-dihydro-1H-inden-1-yl]-3-methylpiperazin-1-yl-4-methylpiperidin-1-yl)carbonyl]-4,6-dimethylpyrimidine; and
iii) 5-[(4-{(3S)-4-[(1R,2R)-2-ethoxy-5-(1,3-thiazol-2-yl)-2,3-dihydro-1H-inden-1-yl]-3-methylpiperazin-1-yl}-4-methylpiperidin-1-yl)carbonyl]-4,6-dimethylpyrimidine; or pharmaceutically acceptable salts thereof.

43. The method of claim 42 wherein said pharmaceutical agents of (A), (B), (C), and (D) are administered together in a pharmaceutical composition provided in a solid dosage form suitable for oral administration.

44. The method of claim 42 wherein said pharmaceutical agents (A), (B), (C), and (D) are administered to said patient once per day.

45. A method for treating an HIV infection in a person, comprising administering to the person, separately or together, therapeutically effective amounts of:
(A) lamivudine;
(B) zidovudine;
(C) efavirenz; and
(D) a CCR5 antagonist.
